(19) United States
(12) Patent Application Publication
(10) Pub. No.: US 2026/0045321 A1
(43) Pub. Date: Feb. 12, 2026

(54) METHOD AND SYSTEM FOR SIMULATING STEM CELL DIFFERENTIATION DYNAMICS

(71) Applicant: CellVoyant Technologies Limited, Bristol (GB)

(72) Inventors: SAAD MOHAMMAD, Bristol (GB); ROBERT VAN DIJK, Bristol (GB); RICKARD SJÖGREN, Bristol (GB)

(21) Appl. No.: 19/290,563

(22) Filed: Aug. 5, 2025

Related U.S. Application Data
(60) Provisional application No. 63/679,714, filed on Aug. 6, 2024.

(51) Int. Cl. G16B 45/00; G06N 3/0455; G06N 3/08; G16B 40/00

(52) U.S. Cl. CPC G16B 45/00; G06N 3/0455; G06N 3/08; G16B 40/00

(57) ABSTRACT

Provided herein are computer-implemented methods for cell culture representation, non-transitory computer readable storage mediums for storing one or more programs associated with cell culture representation, and systems for cell culture representation.

### CROSS REFERENCE TO RELATED APPLICATION

This application claims priority to U.S. Provisional Application No. 63/679,714, filed Aug. 6, 2024, the disclosure of which is herein incorporated by reference in its entirety.

### TECHNICAL FIELD

The present disclosure relates generally to a system, an apparatus, and a computer-implemented method for learning a representation of cell culture biology.

### BACKGROUND

Stem cell differentiation efficiency exhibits a notable degree of inconsistency among different cell lines and donors. For instance, gene expression and methylation differ between lines. This inconsistency has prompted researchers to assess the shortcomings of traditional static differentiation protocols, which often yield suboptimal results due to their inherent inflexibility.

To address this issue, there is growing interest in the concept of dynamic differentiation protocols. These protocols, in theory, involve the adjustment of timings and concentrations of added growth factors based on the current state of the cell culture. This may lead to improved outcomes, although the exact efficacy remains uncertain.

An unmet need exists for an analytical solution that is inexpensive, fast, and can provide detailed insight into biology to provide an optimal foundation for implementing dynamic protocols.

### SUMMARY

The instant disclosure provides an innovative technological solution that learns a numeric representation of a cell culture process by a machine learning platform, such as, for example, an artificial neural network (ANN), using two or more different types of measurements of a given cell culture. In various embodiments, the measurements can be taken from different cell cultures, so long as some measurements are aligned. The different types of measurements can include complementary types of measurements that can show different facets of an underlying biology. The measurements can include, for example, live cell microscopic imaging complemented by immunofluorescent imaging of the cell culture, among other types of measurements. For each measurement type, multiple measurements can be taken over time and measurement data collected. Once trained, the machine learning platform can be used to forecast the state of the cell culture at any point in time in the future, as provided forecasted measurement data for the culture at such time. For instance, a trained multi-modal model can reconstruct at least two sets of measurements obtained from the same process that overlap partially with respect to covariates, such as, for instance, time, cell line, and process conditions. Through compact joint representation, forecasting can be greatly simplified. The solution can perform cross prediction, thereby requiring only one of a plurality of modalities to infer another one of the modalities, since the solution can pick the most efficient and easily accessible modality at inference time.

The technological solution can solve, among others, the problem of predicting an outcome of a downstream stage based on cell culture process data. The solution is designed to handle the challenging number of datapoints that carry through multi-stage processes, such as, for example, in making cell therapies, and decrease at every stage of the process. The solution is designed to handle cases in which there are more vessels being cultured than are later released and quality controlled due to drop-out at every stage. While plentiful data can be found during, for example, drug and process development, where cell cultures are repeated many times, the number of cell culture runs that later map 1:1 to clinical administration are, however, very few. The disclosed solution is designed to overcome, among other things, the problem of predicting a later stage outcome based on cell culture data, even in the absence of large datasets that would otherwise be necessary.

The instant disclosure provides a solution that facilitates faster development and lower risk manufacturing in making cell therapies, which are typically long and complex multi-step processes with multiple successive stages, each of which depends on earlier stages and the interactions. The solution includes systems and methodologies that can predict the success of later stages during earlier, for example, clinical outcomes such as during cell culturing, thereby greatly facilitating faster development and lower risk manufacturing.

According to an aspect of the disclosure, a computer-implemented method is provided for cell culture representation based on two or more modalities. The methods comprise receiving, by a processor, a first set of measurements of a cell culture, the first set of measurements being a first type of measurements; applying, by the processor, the first set of measurements to a machine learning model; predicting, by the machine learning model, a second set of measurements based on the first set of measurements, the second set of measurements being either the first type of measurements or a second type of measurements; and sending, by the processor, the second set of measurements to a computing device. The machine learning model is trained by receiving, by the processor, at least two sets of training measurements of a cell culture, including a first set of training measurements of the first type and a second set of training measurements of the second type, with the second set of training measurements complementing the first set of training measurements; and training the machine learning model by an artificial neural network having a joint embedding space and a plurality of encoder-decoder pairings, including a first encoder-decoder pairing for the first set of training measurements and a second encoder-decoder for the second set of training measurements, wherein the first encoder-decoder pairing comprises a first encoder and a first decoder, and the second encoder-decoder pairing comprises a second encoder and a second decoder.

In certain embodiments, the training the model by the artificial neural network comprises updating encoder-decoder weights of the first encoder-decoder pairing using an autoencoder objective by reconstructing the first set of training measurements by the first encoder-decoder pairing.

In certain embodiments, the training the model by the artificial neural network comprises updating encoder-decoder weights using a predictive cross-encoder objective to encode the first set of training measurements by the first encoder, and decoding a joint embedding by the second decoder, wherein the updating the encoder-decoder weights is based at least in part on a reconstruction loss on the second set of training measurements.

In certain embodiments, the training the model by the artificial neural network comprises updating encoder-decoder weights using a predictive cross-encoder objective to encode the second set of training measurements by the second encoder, and decoding a joint embedding by the first decoder, wherein the updating the encoder-decoder weights is based at least in part on a reconstruction loss on the first set of training measurements.

In certain embodiments, the training the model by the artificial neural network comprises updating encoder-decoder weights of the second encoder-decoder pairing using an autoencoder objective by reconstructing the second set of training measurements by the second encoder-decoder pairing.

In certain embodiments, at least one of the first set of training measurements and/or the second set of training measurements comprises a measurement of the cell culture selected from the group consisting of a transmitted light microscopy image, a fluorescence microscopy image, a phase contrast microscopy image, differential interference contrast microscopy image, polarized light microscopy image, electron microscopy image, structured illumination microscopy image, a pH measurement, a glucose measurement, a lactate measurement, a temperature measurement, a pressure measurement, a dissolved oxygen measurement, a spectroscopy measurement, a conductivity measurement, an optical density measurement, a capacitance measurement, a viscosity measurement, a redox potential measurement, a mass spectroscopy measurement, an ultrasound-based measurement of fluid density, a nutrient measurement, an -omics measurement, and a combination thereof. An -omics measurement can, for example, include a metabolomics measurement, a genomics measurement, an epigenomics measurement, a lipidomics measurement, a glycomics measurement, a transcriptomics measurement, a proteomics measurement, and/or a combination thereof.

In certain embodiments, the cell culture is a stem cell culture undergoing differentiation. The stem cell culture can, for example, be selected from an embryonic stem cell culture, an adult stem cell culture, an induced pluripotent stem cell culture, or a trophoblast stem cell culture. The stem cell culture can, for example, be a mesenchymal stem cell culture, a hematopoietic stem cell culture, a neural stem cell culture, an epithelial stem cell culture, or a cord blood stem cell culture. In certain embodiments, the stem cell culture comprises progenitor cells. The progenitor cells can, for example, be selected from the group consisting of mesodermal progenitor cells, endodermal progenitor cells, ectodermal progenitor cells, neural progenitor cells, cardiac progenitor cells, hematopoietic progenitor cells, mesenchymal stem cells, pancreatic progenitor cells, and a combination thereof.

In certain embodiments, the stem cell culture undergoing differentiation results in the stem cell culture differentiating into a mesoderm, endoderm, and/or ectoderm. The mesoderm can, for example, comprise a skeletal muscle cell, a kidney cell, a red blood cell, or a smooth muscle cell. The endoderm can, for example, comprise a lung cell, a thyroid cell, or a pancreatic cell. The ectoderm can, for example, comprise a skin cell, a neuron cell, or a pigment cell.

In certain embodiments, the machine learning model comprises a joint embedded space model.

In certain embodiments, training the machine learning model by the artificial neural network comprises applying a temporal smoothing loss to joint embedding to ensure there is a smooth transition between embeddings of measurements taken at nearby time points. In certain embodiments, training the machine learning model by the artificial neural network comprises applying a regularization to joint embedding to ensure that the embedding is constrained to a multivariate probability distinction.

According to another aspect of the disclosure, a non-transitory computer readable storage medium is provided, storing one or more programs comprising instructions, which, when executed by a processor, perform receiving a first set of measurements of a cell culture, the first set of measurements being a first type of measurements; applying the first set of measurements to a machine learning model; predicting a second set of measurements based on the first set of measurements, the second set of measurements being either the first type of measurements or a second type of measurements; and sending the second set of measurements to a computing device. The machine learning model is trained by receiving, by the processor, at least two sets of training measurements of a cell culture, including a first set of training measurements of the first type and a second set of training measurements of the second type, with the second set of training measurements complementing the first set of training measurements; and training the machine learning model by an artificial neural network having a joint embedding space and a plurality of encoder-decoder pairings, including a first encoder-decoder pairing for the first set of training measurements and a second encoder-decoder for the second set of training measurements, wherein the first encoder-decoder pairing comprises a first encoder and a first decoder, and the second encoder-decoder pairing comprises a second encoder and a second decoder.

In certain embodiments, the training the model by the artificial neural network comprises updating encoder-decoder weights of the first encoder-decoder pairing using an autoencoder objective by reconstructing the first set of training measurements by the first encoder-decoder pairing.

In certain embodiments, the training the model by the artificial neural network comprises updating encoder-decoder weights using a predictive cross-encoder objective to encode the first set of training measurements by the first encoder, and decoding a joint embedding by the second decoder, wherein the updating the encoder-decoder weights is based at least in part on a reconstruction loss on the second set of training measurements.

In certain embodiments, the training the model by the artificial neural network comprises updating encoder-decoder weights using a predictive cross-encoder objective to encode the second set of training measurements by the second encoder, and decoding a joint embedding by the first decoder, wherein the updating the encoder-decoder weights is based at least in part on a reconstruction loss on the first set of training measurements.

In certain embodiments, the training the model by the artificial neural network comprises updating encoder-decoder weights of the second encoder-decoder pairing using an autoencoder objective by reconstructing the second set of training measurements by the second encoder-decoder pairing.

In certain embodiments, at least one of the first set of training measurements and/or the second set of training measurements comprises a measurement of the cell culture selected from the group consisting of a transmitted light microscopy image, a fluorescence microscopy image, a phase contrast microscopy image, differential interference contrast microscopy image, polarized light microscopy image, electron microscopy image, structured illumination microscopy image, a pH measurement, a glucose measurement, a lactate measurement, a temperature measurement, a pressure measurement, a dissolved oxygen measurement, a spectroscopy measurement, a conductivity measurement, an optical density measurement, a capacitance measurement, a viscosity measurement, a redox potential measurement, a mass spectroscopy measurement, an ultrasound-based measurement of fluid density, a nutrient measurement, an -omics measurement, and a combination thereof. An -omics measurement can, for example, include a metabolomics measurement, a genomics measurement, an epigenomics measurement, a lipidomics measurement, a glycomics measurement, a transcriptomics measurement, a proteomics measurement, and/or a combination thereof.

In certain embodiments, the cell culture is a stem cell culture undergoing differentiation. The stem cell culture can, for example, be selected from an embryonic stem cell culture, an adult stem cell culture, an induced pluripotent stem cell culture, or a trophoblast stem cell culture. The stem cell culture can, for example, be a mesenchymal stem cell culture, a hematopoietic stem cell culture, a neural stem cell culture, an epithelial stem cell culture, or a cord blood stem cell culture. In certain embodiments, the stem cell culture comprises progenitor cells. The progenitor cells can, for example, be selected from the group consisting of mesodermal progenitor cells, endodermal progenitor cells, ectodermal progenitor cells, neural progenitor cells, cardiac progenitor cells, hematopoietic progenitor cells, mesenchymal stem cells, pancreatic progenitor cells, and a combination thereof.

In certain embodiments, the stem cell culture undergoing differentiation results in the stem cell culture differentiating into a mesoderm, endoderm, and/or ectoderm. The mesoderm can, for example, comprise a skeletal muscle cell, a kidney cell, a red blood cell, or a smooth muscle cell. The endoderm can, for example, comprise a lung cell, a thyroid cell, or a pancreatic cell. The ectoderm can, for example, comprise a skin cell, a neuron cell, or a pigment cell.

In certain embodiments, the machine learning model comprises a joint embedded space model.

In certain embodiments, training the machine learning model by the artificial neural network comprises applying a temporal smoothing loss to joint embedding to ensure there is a smooth transition between embeddings of measurements taken at nearby time points. In certain embodiments, training the machine learning model by the artificial neural network comprises applying a regularization to joint embedding to ensure that the embedding is constrained to a multivariate probability distinction.

According to a further aspect of the disclosure, a system for cell culture representation based on two or more modalities is provided. The system can, for example, comprise a memory; a communication unit configured to receive requests from, or send forecasting measurements to, a computing device; and a processor, wherein the processor is configured to receive at least two sets of training measurements of a cell culture, including a first set of training measurements of the first type and a second set of training measurements of the second type, with the second set of training measurements complementing the first set of training measurements; and train a machine learning model by an artificial neural network having a joint embedding space and a plurality of encoder-decoder pairings, including a first encoder-decoder pairing for the first set of training measurements and a second encoder-decoder for the second set of training measurements, wherein the first encoder-decoder pairing comprises a first encoder and a first decoder, and the second encoder-decoder pairing comprises a second encoder and a second decoder.

In certain embodiments, the training the model by the artificial neural network comprises updating encoder-decoder weights of the first encoder-decoder pairing using an autoencoder objective by reconstructing the first set of training measurements by the first encoder-decoder pairing.

In certain embodiments, the training the model by the artificial neural network comprises updating encoder-decoder weights using a predictive cross-encoder objective to encode the first set of training measurements by the first encoder, and decoding a joint embedding by the second decoder, wherein the updating the encoder-decoder weights is based at least in part on a reconstruction loss on the second set of training measurements.

In certain embodiments, the training the model by the artificial neural network comprises updating encoder-decoder weights using a predictive cross-encoder objective to encode the second set of training measurements by the second encoder, and decoding a joint embedding by the first decoder, wherein the updating the encoder-decoder weights is based at least in part on a reconstruction loss on the first set of training measurements.

In certain embodiments, the training the model by the artificial neural network comprises updating encoder-decoder weights of the second encoder-decoder pairing using an autoencoder objective by reconstructing the second set of training measurements by the second encoder-decoder pairing.

In certain embodiments, at least one of the first set of training measurements and/or the second set of training measurements comprises a measurement of the cell culture selected from the group consisting of a transmitted light microscopy image, a fluorescence microscopy image, a phase contrast microscopy image, differential interference contrast microscopy image, polarized light microscopy image, electron microscopy image, structured illumination microscopy image, a pH measurement, a glucose measurement, a lactate measurement, a temperature measurement, a pressure measurement, a dissolved oxygen measurement, a spectroscopy measurement, a conductivity measurement, an optical density measurement, a capacitance measurement, a viscosity measurement, a redox potential measurement, a mass spectroscopy measurement, an ultrasound-based measurement of fluid density, a nutrient measurement, an -omics measurement, and a combination thereof. An -omics measurement can, for example, include a metabolomics measurement, a genomics measurement, an epigenomics measurement, a lipidomics measurement, a glycomics measurement, a transcriptomics measurement, a proteomics measurement, and/or a combination thereof.

In certain embodiments, the cell culture is a stem cell culture undergoing differentiation. The stem cell culture can, for example, be selected from an embryonic stem cell culture, an adult stem cell culture, an induced pluripotent stem cell culture, or a trophoblast stem cell culture. The stem cell culture can, for example, be a mesenchymal stem cell culture, a hematopoietic stem cell culture, a neural stem cell culture, an epithelial stem cell culture, or a cord blood stem cell culture. In certain embodiments, the stem cell culture comprises progenitor cells. The progenitor cells can, for example, be selected from the group consisting of mesodermal progenitor cells, endodermal progenitor cells, ectodermal progenitor cells, neural progenitor cells, cardiac progenitor cells, hematopoietic progenitor cells, mesenchymal stem cells, pancreatic progenitor cells, and a combination thereof.

In certain embodiments, the stem cell culture undergoing differentiation results in the stem cell culture differentiating into a mesoderm, endoderm, and/or ectoderm. The mesoderm can, for example, comprise a skeletal muscle cell, a kidney cell, a red blood cell, or a smooth muscle cell. The endoderm can, for example, comprise a lung cell, a thyroid cell, or a pancreatic cell. The ectoderm can, for example, comprise a skin cell, a neuron cell, or a pigment cell.

In certain embodiments, the machine learning model comprises a joint embedded space model.

In certain embodiments, the artificial neural network comprises an encoder suite comprising a plurality of encoders, including the first encoder and the second encoder. In certain embodiments, the artificial neural network comprises a decoder suite comprising a plurality of decoders, including the first decoder and the second decoder. The number of encoders in the encoder suite can, for example, equal the number of decoders in the decoder suit.

In certain embodiments, the joint embedding space is configured to receive encoded measurement data from each of the plurality of encoders; and each of the plurality of decoders is configured to receive joint embedded data from the joint embedding space to reconstruct at least one of the first set of training data and the second set of training data.

In certain embodiments, training the machine learning model by the artificial neural network comprises applying a temporal smoothing loss to joint embedding to ensure there is a smooth transition between embeddings of measurements taken at nearby time points. In certain embodiments, training the machine learning model by the artificial neural network comprises applying a regularization to joint embedding to ensure that the embedding is constrained to a multivariate probability distinction.

Additional features, advantages, and embodiments of the disclosure may be set forth or apparent from consideration of the detailed description and drawings. Moreover, it is to be understood that the foregoing summary of the disclosure and the following detailed description and drawings provide non-limiting examples that are intended to provide further explanation without limiting the scope of the disclosure as claimed.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure. No attempt is made to show structural details of the disclosure in more detail than may be necessary for a fundamental understanding of the disclosure and the various ways in which it may be practiced.

FIG. 1 shows a block diagram of a system for learning a joint abstract representation of biology.

FIG. 2 shows a high-level diagram of an operation of a cell culture representation system to train a multi-modal model.

FIG. 3 shows a high-level diagram of an operation by the cell culture system to perform self-reconstruction on all sets of measurements, as well as cross-reconstruction on matching measurements of other modalities.

FIG. 4 shows a nonlimiting example of the cell culture system predicting, by the trained multi-modal embedding model, a second measurement modality.

FIG. 5 shows a nonlimiting example of how the cell culture representation system can train and use the multi-modal model to learn a joint embedding over different cell culture vessels.

FIG. 6A shows a non-limiting example of a cell culture system predicting, by the trained embedding model, a downstream measurement based on embedded cell culture measurements as input.

FIG. 6B shows a nonlimiting example of a process that can be performed by the cell culture representation system.

FIG. 7 shows another nonlimiting example of a process that can be performed by the cell culture representation system.

FIG. 8 shows a nonlimiting embodiment of the cell culture representation system, constructed according to the principles of the disclosure.

FIG. 9 shows an example of the prediction results. The input consists of live phase contrast images from multiple fields, while the output is the probability distribution of binned immunofluorescence intensity for the Brachyury mesoderm marker. The figure illustrates predictions at two different time points. The first time point, 4 hours, represents live phase contrast images where the cell culture has not yet been fixed, making ground truth measurement impossible. The second time point, 140 hours, corresponds to the end of day 6. At this point, the cells were fixed and treated with immunofluorescence, providing the ground truth for comparison.

FIG. 10 shows a plate layout of all replicate plates. Cell culture conditions differ per row based on the target fate, listed on the left of the figure. Phase contrast images are taken in each well, the Oct 4 pluripotent marker was visualized using immunofluorescence imaging on even columns and the ectoderm (Sox 1) and mesoderm (Brachyury) markers were visualized on the odd columns. Every two columns a different cell line was plated.

FIG. 11 shows an overview of the method illustrating prediction application. The image depicts the key components of the method, including self-reconstruction and cross-reconstruction losses, as well as the KL divergence loss. The self-reconstruction loss ensures accurate reconstruction within each modality (modality 1 to modality 1, and modality 2 to modality 2). The cross-reconstruction loss enables reconstruction across modalities when their covariates are aligned (from modality 1 to modality 2 and vice versa). Additionally, a KL divergence/entropy regularization loss is applied to optimize the uncertainty between the multi-modal embeddings. This regularization is crucial due to differing levels of information across modalities, such as between image and histogram measurements. The wider distribution (higher entropy) of the embedding when inputting a histogram reflects greater uncertainty in reconstructing an image, compared to the reverse situation where image data provides more detailed information. This approach effectively manages the diverse levels of overlapping information across modalities.

FIG. 12 shows a graph demonstrating the comparison of Brachyury-positive cell percentages. This figure compares the predicted versus actual (ground truth) percentages of cells expressing the Brachyury marker, indicating differentiation into mesoderm, across three different cell lines over time. The cells were specifically differentiated into mesodermal lineage. The comparison highlights the accuracy of the predictions in capturing the dynamic changes in marker expression throughout the mesoderm differentiation process.

FIGS. 13A-13E show graphs showing the forecasting results using multi-field phase contrast images. The figure illustrates forecasting results based on multi-field live phase contrast images taken at 4, 8, and 12 hours. These images were used to predict the Brachyury-marker intensity distribution up to 132 hours into the future (from the last input at 12 h to the last GT time-point at 144 h), and the predicted distributions at 24, 48, 96, 120 and 144 h are compared with the corresponding GT data. This highlights the model's accuracy in long-term forecasting of the marker's distribution.

FIG. 14 shows a graph showing Jensen-Shannon Divergence (JSD) error between the forecasted and actual intensity distributions of the Brachyury marker. The data for forecasting were derived from multi-field live phase contrast images taken at 4, 8, and 12 hours, which were used to project the Brachyury marker intensity distributions up to 140 hours into the future. The curves represent JSD errors at various time points, specifically at 24, 48, 72, and 140 hours, where ground truth immunofluorescence (IF) intensity distributions are available. Each curve illustrates the accuracy of the forecasts relative to the actual marker intensity distributions across three different cell lines over time, highlighting the performance of the forecasting model in capturing long-term changes in the Brachyury marker distribution.

FIG. 15 shows box plots comparing SKLD scores between the CNN-LSTM (black) and DFE-MMoE (grey) approach across cell lines. Each data point represents the mean SKLD score computed for each unique combination of cell line, hour, and marker. Wilcoxon signed-rank tests are computed between the two Method distributions to assess significance indicated by a*(p≤0.05) or ns (not significant), highlighting a significant improvement of proposed method over commonly used methods (CNN-LSTM) for ⅔ cell lines (or ⅔ use cases).

FIG. 16 shows box plots comparing SKLD scores between the CNN-LSTM (black) and DFE-MMoE (grey) approach across different days expressed in hours. Each data point represents the mean SKLD score computed for each unique combination of stem cell line, hour, and marker. Wilcoxon signed-rank tests are computed between the two method distributions to assess significance indicated by a*(p≤0.05) or ns (not significant), highlighting the capacity of the proposed method to significantly improve upon commonly used methods (CNN-LSTM) in long-term (>24 hours) non-linear dynamics.

FIG. 17 shows box plots comparing SKLD scores between the CNN-LSTM (black) and DFE-MMoE (grey) approach across different markers. Each data point represents the mean SKLD score computed for each unique combination of cell line, hour, and marker. Wilcoxon signed-rank tests are computed between the two method distributions to assess significance indicated by a*(p≤0.05) or ns (not significant), highlighting significant improvement over commonly used methods (CNN-LSTM) for ⅓ markers predicted.

The present disclosure is further described in the detailed description that follows.

### DETAILED DESCRIPTION

The disclosure and its various features and advantageous details are explained more fully with reference to the non-limiting embodiments and examples that are described or illustrated in the accompanying drawings and detailed in the following description. It should be noted that features illustrated in the drawings are not necessarily drawn to scale, and features of one embodiment can be employed with other embodiments as those skilled in the art would recognize, even if not explicitly stated. Descriptions of well-known components and processing techniques may be omitted so as to not unnecessarily obscure the embodiments of the disclosure. The examples are intended merely to facilitate an understanding of ways in which the disclosure can be practiced and to further enable those skilled in the art to practice the embodiments of the disclosure. Accordingly, the examples and embodiments should not be construed as limiting the scope of the disclosure. Moreover, it is noted that like reference numerals represent similar parts throughout the several views of the drawings.

There exists a range of analytical approaches to characterize a cell culture process. These analytical techniques could be simple measurements such as pH, glucose or lactate concentration, or dissolved oxygen that can be performed in real-time with high frequency. Intermediate complexity instruments such as label-free imaging allow real-time measurements, albeit with lower measurement frequency, and capture more nuanced biology by enabling measurements of cell size and morphology. Richest insight into biology is acquired by, potentially destructive, measurements such as flow cytometry, immunocytochemistry, -omics (such as, e.g., metabolomics, genomics, epigenomics, lipidomics, glycomics, lipidomics, transcriptomics, and proteomics), albeit at higher cost and lower throughput compared to the simpler alternatives.

Ideally, an analytical technique should be cheap, fast, and enable detailed insight into biology to provide an optimal foundation for implementing dynamic protocols. Since no such technology exists to date, a combination of analytical technologies is commonly used to characterize cell culture processes.

It is possible to use machine learning to learn an abstract representation of an underlying biology using data from different analytical technologies. However, standard supervised machine learning requires paired measurements, where two measurements are acquired under the same conditions (for instance at the same time point). In real-life processes, the number of paired measurements is far smaller than the total number of measurements. Using standard machine learning would then mean that much data, and hence information, is left on the table.

A common approach to reduce variability in cell culture processes is to purify target or progenitor cells from other cell types. This has for instance been done in for example neural progenitor cells and foregut endoderm cells. This approach, however, introduces additional complexity in the protocol by needing an explicit purification stage. Implementing purification correctly requires method development on its own.

Another approach described in literature uses live cell imaging and machine learning. This approach provides a set of specialized machine learning models and needs additional method development for each new use case.

Further approaches involve directly controlling the gene expression of certain genes based on fluorescence readout. Such approaches have been demonstrated for both maintaining embryonic stem cell culture as well as improving differentiation. These approaches, however, rely on being able to directly measure gene expression of key genes by using genetically engineered cells and are therefore not easily scalable to new use cases.

State-of-the-art solutions that inform how to run a bioprocess fall into two main categories. The first main category is based on growth modelling, whereas the population growth and death dynamics are modelled and forecasted. These models can be mechanistic, where the dynamics are described using differential equations, or data-driven, such as machine learning models. There are also hybrid models, that use a combination of mechanistic and data-driven models. Hybrid models can be found in biomanufacturing of antibodies but have been demonstrated in cell therapy manufacturing too. These growth models provide no insight into how cell state changes during differentiation due to the poor understanding of how process parameters link to differentiation processes.

The second main category models cell state changes on a molecular level using equation systems based on gene regulatory networks. Simpler approaches based on the expression of single transcription factors have been demonstrated in control of cell culture processes, such as, for example, in maintaining embryonic stem cell culture and improving differentiation. These approaches are limited to modelling cellular states where the gene regulatory network is well understood. Use during real-time forecasting of a cell culture process also relies on being able to directly measure gene expression of key genes, such as, for instance, by using genetically engineered cells with fluorescent markers. They are therefore not easily scalable to new use cases.

The instant disclosure provides a technological solution that solves the problem of effectively learning a joint abstract representation of biology using data from different analytical technologies where paired measurements are sparse. The technological solution includes a method for training a multi-modal model that learns an abstract representation of a cell culture process. The trained multi-modal model can reconstruct at least two sets of measurements obtained from the same process that overlap partially with respect to covariates, such as, for instance, time, cell line, and process conditions.

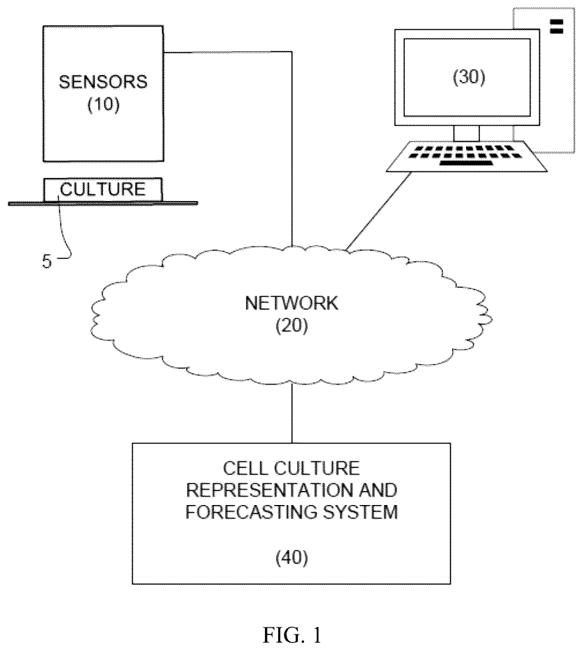

FIG. 1 Shows a block diagram of a system for learning a joint abstract representation of biology. The system includes one or more sensors 10, a network 20, a computing device 30, and a cell culture representation and forecasting (CCRAF) system 40. In various embodiments, the sensors 10 can include a variety of sensor devices, each configured to monitor and measure a different attribute of a cell culture process, including the attributes and real-time conditions of individual cells, cell types, cell subtypes, and fates in the culture, as well as time, cell lines, and process conditions.

Measurement data received from the sensors 10 by the CCRAF system 40, such as, for example, via the network 20, can be processed and used to train a multi-modal model to learn an abstract representation of a culture process for a given cell culture 5. Measurement data can also be received by the CCRAF system 40 from the computing device 30, which can include measurement data input, for example, by a human operator using a human user interface (UI). Once trained, the model can be used by the CCRAF system 40 to forecast the state and conditions of a given cell culture at any point in time in the future, including predicting measurement data and any combination of measurements at such time.

The sensors 10 can include one or more sensor devices configured to measure attributes such as inline or online process parameters, including at least one of pH, glucose, lactate, temperature, dissolved oxygen, spectroscopy (RAMAN, Near infrared, Fourier transform infrared), conductivity for ionic strength and composition, optical density for biomass, capacitance for viable cell density, medium viscosity, redox potential for metabolic state and cell stress, mass spectrometry, and ultrasound-based measurement of fluid density.

The sensors 10 can include one or more sensor devices configured to measure attributes such as online or atline or offline phenotypic parameters, including at least one of imaging and flow cytometry.

The sensors 10 can include at least one image pickup device, which can include, for example, a two-dimensional (2D) or three-dimensional digital image pickup device, such as, for example, a digital microscope camera, an electron microscope, or other high or ultrahigh resolution microscopic image pickup device (such as, for example, 1.5, 5, 10, 12, or 18 megapixels, or greater).

The sensors 10 can include a plurality of image pickup devices, at least one of which is configured to capture live cell images of the culture, and at least one other of which is configured to capture immunofluorescent images of the culture.

Each sensor 10 can be configured to monitor and measure an attribute of the cell culture and send the measurement data via a communication link to the computing device 30 or the cell culture simulator 40, including, for example, pH measurements, glucose measurements, lactate measurements, temperature measurements, pressure measurements, dissolved oxygen measurements, spectroscopy measurements, conductivity measurements, optical density measurements, capacitance measurements, medium viscosity measurements, redox potential measurements, mass spectrometry measurements, ultrasound-based measurements of fluid density, nutrient measurements, -omics measurements (e.g., metabolomics, genomics, epigenomics, lipidomics, glycomics, transcriptomics, and/or proteomics), image data (e.g., transmitted light microscopy images, fluorescence microscopy images, phase contrast microscopy images, differential interference contrast microscopy images, and/or polarized light microscopy images), and flow cytometry data. The measurement data can be sent directly, or via the network 20, to the computing device 30 or the CCRAF system 40.

The computing device 30 can be configured to render (such as, for example, display) cell culture data, including measurement data representative of the current cell state and the cell state at any time in the future, action(s), as well as forecasted measurements of one or more measurement types at any time in the future. The information can be rendered by a human interface device, such as, for example, a graphic user interface (GUI) on a display device, an interactive voice response (IVR) unit and speaker, or other communication signal perceivable by a human user. The computing device 30 is further configured to receive annotations for each cell culture and type of measurement, including, for example, a label and description for each cell type, subtype, fate, or other feature of the cell culture, and each type of measurement. The computing device 30 can be configured to communicate and interact with the sensors 10 and the CCRAF system 40 via one or more communication links.

The CCRAF system 40 is configured to receive real-time measurement data from the sensors 10 or computing device 30 and store historical measurement data for each cell culture and each type of measurement. The CCRAF system 40 is configured to analyze, in real-time, the measurement data and historical data, including one or more types of measurements of a cell culture, and forecast the future state of the cell culture undergoing differentiation based on the current state of the cell culture, as well as measurements for the cell culture at that the time of the future state. The CCRAF system 40 includes a machine learning (ML) platform having one or more ML models that can be trained as described herein.

The CCRAF system 40 is configured to communicate with the sensors 10 and the computing device 30 and receive measurement data, including two or more different types of measurements. The CCRAF system 40 is also configured to communicate with the computing device 30 and receive one or more forecast requests, including requests for measurement predictions for a cell culture, and date/time information for a time point in the future for a particular cell culture.

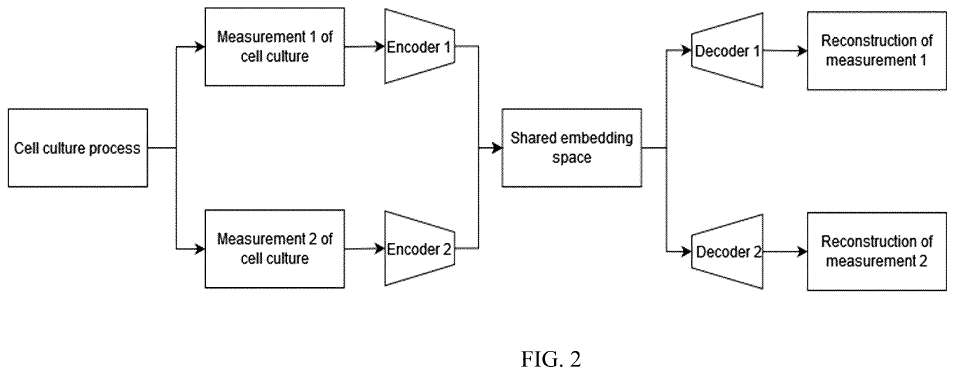

FIG. 2 depicts a high-level flow diagram of an operation of the CCRAF system 40 to train a multi-modal model, according to the principles of the disclosure. As seen, the CCRAF system 40 can include encoder-decoder pairings for each type of measurement data and a shared embedding space for joint embedding the two or more encoder-decoder pairings. The CCRAF system 40, via the shared embedding space, can map two or more different types of measurement data into a common vector space to build and train encoder-decoder models that can understand the relationships between these two or more types of measurement data and make predictions based on the learned understanding.

Referring to FIGS. 1 and 2, the first and second sets of measurements of a cell culture process can be obtained from, for example, from the sensors 10 or computing device 30, and fed to respective first and second encoders in the CCRAF system 40. The second set of measurements can be of a different measurement type from the first set of measurements, with the second set of measurements complementing the first set of measurements. In at least one embodiment, the first set of measurement data includes live cell microscopic images of the cell culture and the second set of measurement data includes immunofluorescent images of the cell culture, which complement the microscopic images. In various embodiments the first set of measurement data and the second set of measurement data can include partially matched data.

After encoding by the first and second encoders (Encoder 1 and Encoder 2, respectively) the respective first and second sets of measurement data, the encoded data can be fed to the shared embedding space for joint embedding to map the first and second sets of measurement data (Measurement 1 and Measurement 2, respectively) into a common vector space and, with decoding by the respective first and second decoders (Decoder 1 and Decoder 2), to build and train encoder-decoder models that can understand the relationships between these two or more types of measurement data and make predictions based on the learned understanding. The model(s) can then be used by the CCRAF system 40 to reconstruct the first and/or second measurements of the cell culture process.

An important feature of the CCRAF system 40 lies in its training procedure that eliminates the need for different sets of measurements to overlap completely. By training the encoder-decoder models to perform self-reconstruction on all sets of measurements, as well as cross-reconstruction on matching covariates, the CCRAF system 40 can train a joint embedding space (JES) model with many uses.

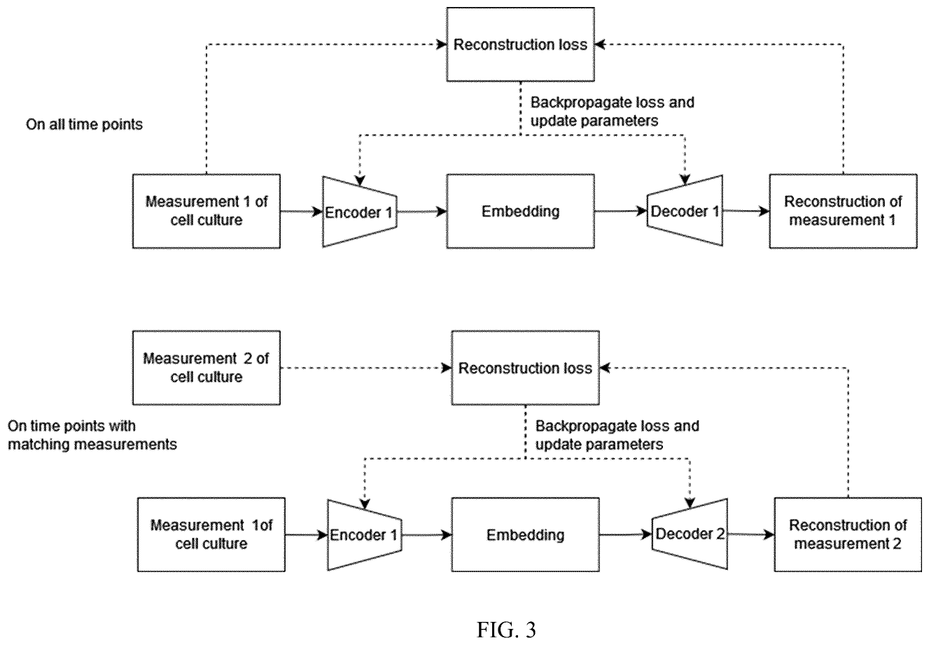

FIG. 3 shows a high-level diagram of an operation by the CCRAF system 40 to perform self-reconstruction on all sets of measurements, as well as cross-reconstruction on matching covariates. Using time as an example covariate, in which two sets of time-resolved measurements overlap on a subset of all timepoints, as seen in FIG. 3, the CCRAF system 40 can perform training to obtain a joint multi-modal embedding space model from the first set of measurements (Measurement 1) of the cell culture. The operation seen in the upper part of the Figure shows the training on all time points, including reconstruction of loss and backpropagating loss to update parameters in the first encoder-decoder model. The reconstruction loss can be based on one or both the first set of measurements of the cell culture and the reconstruction of the first set of measurements, thereby tuning the parametric values of the first encoder-decoder model (Encoder 1-Decoder 1) for accuracy.

In at least one embodiment, the second encoder-decoder model (Encoder 2-Decoder 2) can be built and trained on all time points according to the process shown for the first encoder-decoder model in FIG. 3, except that the data input to, and reconstructed by, the second encoder-decoder model is the second set of measurements (Measurement 2) of the cell culture.

The operation seen in the lower part of FIG. 3 includes training on time points with matching measurements for the first and second sets of measurements (Measurement 1 and Measurement 2, respectively) of the cell culture. In this process, the reconstruction loss is based on both the second set of measurements of the cell culture and the reconstruction of the second set of measurements, with the propagated loss being fed back to update parameters in the joint-embedding space (JES) model, thereby tuning the parametric values of the JES model for accuracy. After training the JES model is completed, it is possible to predict any combination of measurements given any measurement.

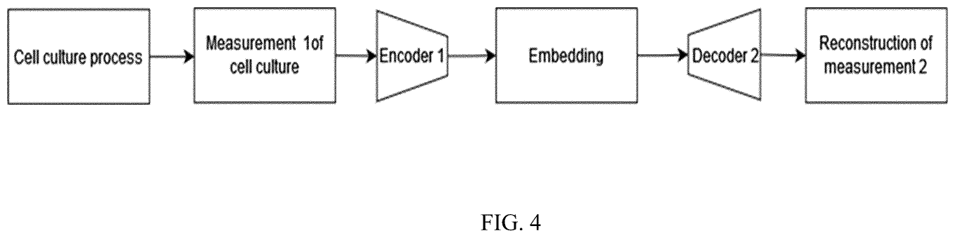

FIG. 4 shows a nonlimiting example of the CCRAF system 40 predicting, by the trained multi-modal embedding (or JES) model, the second measurement modality. By aligning measurements on covariates such as cell line and/or genetic reporters (or other vessel independent covariates), the CCRAF system 40 can perform the cell culture process in two different vessels and learn a joint biological representation over both to build and train a JES model that can then accurately predict at any time in the future any combination of measurements for a given measurement.

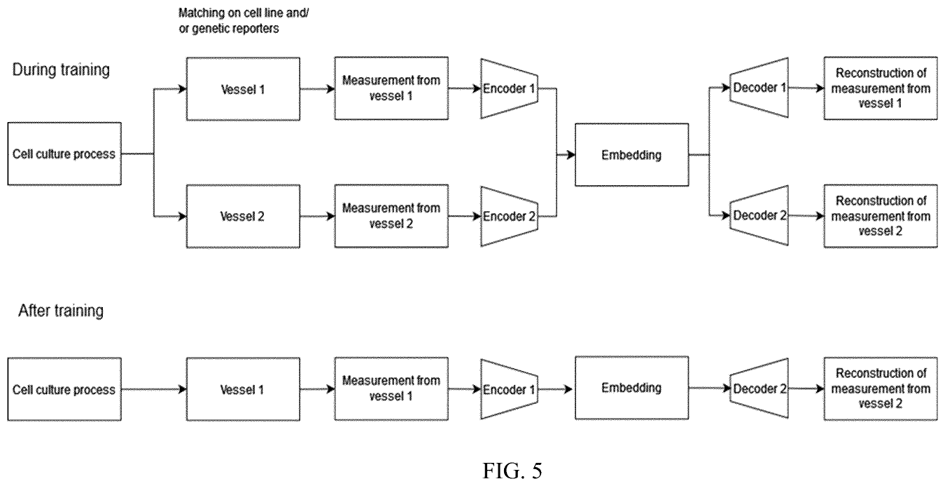

FIG. 5 shows a nonlimiting example of how the CCRAF system 40 can train and use the multi-modal JES model to learn a joint embedding over different cell culture vessels. Differentiation into the same target cell can be done in both lab-scale (such as, for example, in 96-well plates) and small bioprocess scale (such as, for example, in a small bioreactor). The lab-scale process allows for a much larger sample size and more comprehensive measurements due to the lower cost associated with it. By learning a joint representation between the lab-scale and process measurements and building and training the JES model, the CCRAF system 40 can recapitulate some of the information from lab-scale in the bioprocess scale allowing for better informed process decisions.

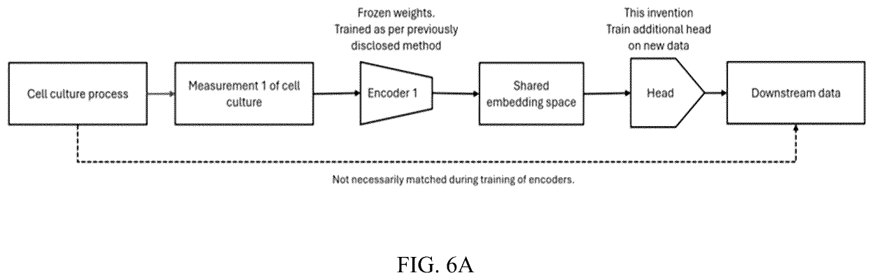

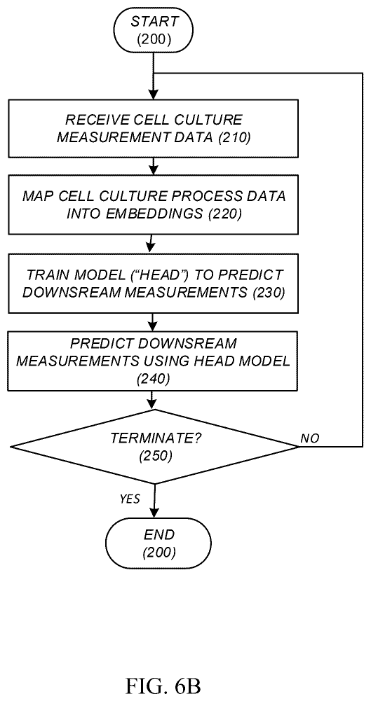

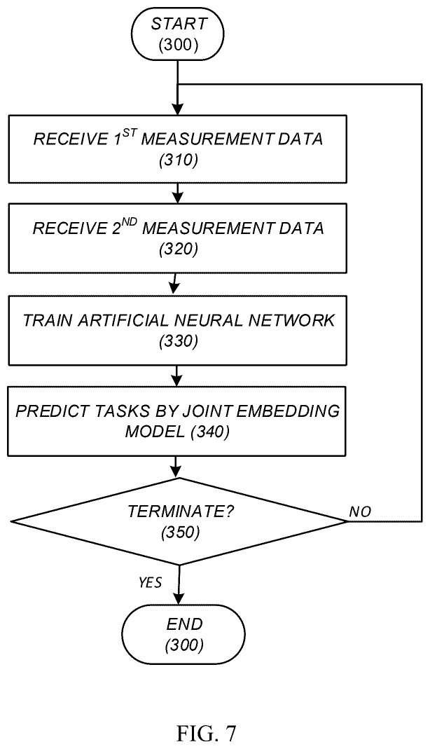

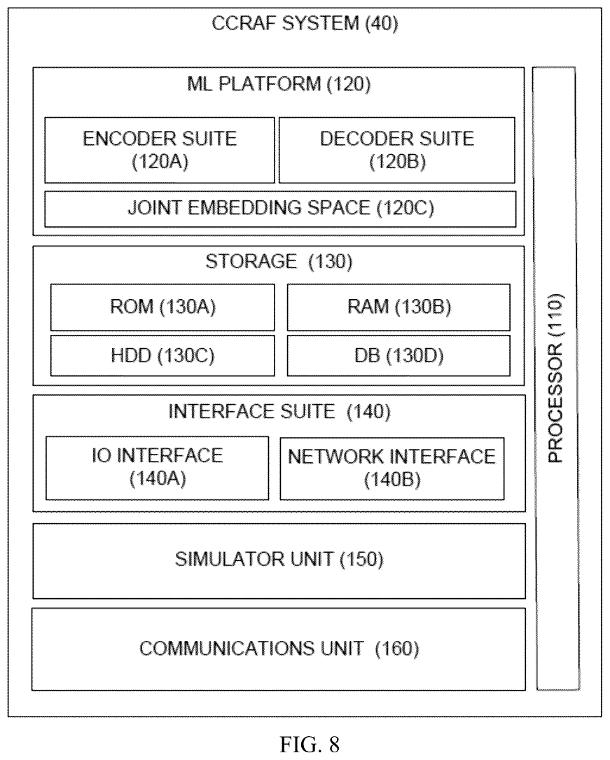

FIG. 6A shows a non-limiting example of the ML platform 120 (in the CCRAF system 40, in FIG. 8) predicting, by the trained multi-modal embedding (or JES) model, a downstream measurement based on embedded cell culture measurements as input. In various embodiments the process depicted in FIG. 6A can be provided as an add-on, in which the ML platform can benefit from the numerical representation learned by the JES model, for example, as described above with reference to FIG. 2, to predict downstream data in a data-efficient manner. In certain embodiments, the process depicted in FIG. 6A can be performed at, or prior to, Step 340 (predict tasks by joint embedding model) in FIG. 7.

In various embodiments the encoders, decoders, or encoder-decoder architectures can each include a unique model, and any combination of the foregoing can include one or more system models. The models can be configured to operate in parallel, cascade, or various combinations of parallel and cascade. Each model can include a trained mathematical function resulting from training the artificial neural network (or ANN) on data as described herein, including in which each trained mathematical function maps inputs to outputs and involves sequence-to-sequence transformations.

Referring to FIGS. 6A and 8, after the JES model is trained (including, for example, according to the process depicted in FIG. 2) to learn a numerical representation of cell culture process data, the ML platform 120 can be trained to predict downstream data. In this regard, the CCRAF system 40 can be configured to receive (for example, via the communications unit 160, in FIG. 8) a collected dataset of cell culture measurements that is matched, for example, 1:1, with measurements of a process stage downstream of the cell culture process. The ML platform 120 can encode the new cell culture measurements into the embedding space using a previously trained encoder in the encoder suite 120A (for example, trained according to the process in FIG. 2), and provide an abstract representation of the cell culture biology with a tensor representation. The ML platform 120 can take the embedded cell culture measurements as input and the downstream measurements as output and train a machine learning model, referred to as Head in FIG. 6A. It is noted that the JES model need not be trained in this stage, its model weights can be “frozen.” Using the trained JES model and the Head on measurements collected in future cell culture processes, the ML platform 120 can predict the outcome of downstream processes at any time in the future.

FIG. 6B shows a nonlimiting example of a process 200 that can be performed by the CCRAF system 40. Initially, measurements of a cell culture process can be performed by the sensor 10 (shown in FIG. 1) and cell culture measurement data received (for example, via the communications unit 160) from the sensors 10 by the ML platform 120 (shown in FIG. 8) (Step 210). Using a model (for example, the JES model) that was previously trained on one or more datasets where at least one of the measurements match to what will be matched to downstream data, the ML platform 120 can apply the received cell culture measurement data to the model to map the cell culture process data into embeddings (Step 220). Using the training process depicted in FIG. 6A, the model “Head” can be trained based on high-dimensional tensor (embeddings from, for example, the process in FIG. 2) as input to predict downstream measurements at a time in the future (Step 240). With the Head model trained, the ML platform 120, or more specifically the Head model, can predict downstream measurements based on future cell culture measurement data (Step 240). Step 210 to 240 can be repeated until a termination request is detected, or the process terminates according to other predefined conditions.

In various embodiments the Head model can be any predictive machine learning model suitable for the type of downstream measurements being collected. It may be a simple logistic regression model predicting a binary success/fail of a later quality control (QC) stage. It may be a artificial neural network predicting a more complex measurement.

In various applications, the JES model is trained to learn a shared representation between multiple types of measurements and, being multi-modal, it learns a complex representation of cell culture biology to do that well. The resulting embedding space is “rich”, meaning that much biological information is encoded into the high-dimensional tensors that are provided by the JES model (for example, the encoders in the encoder suite 120A, as discussed above with respect to FIG. 2). All information that the models can learn at this earlier stage, is information that can be re-used later and not learnt of models trained on top of these embeddings. Models trained on the embeddings can therefore be trained on fewer data points, as much biological information is already available.

FIG. 7 shows another nonlimiting example of a process 300 that can be performed by the CCRAF system 40. The process 300 can be performed by the CCRAF system 400 prior to the process 200 (shown in FIG. 6B). Referring to FIGS. 1 and 7 contemporaneously, a first set of measurements of one or more samples of the cell culture 5 can be obtained, via the sensors 10, by the CCRAF system 40 (Step 310). The first set of measurement data can include, for example, phase contrast images of the cell culture picked up by one of the sensors 10 (for example, an image pickup device). The first set of measurement data can be collected for a multitude of samples, varying the process covariates (for example, time point).

A second set of measurements (for example, complementing the first set of measurements) of the cell culture 5 can be obtained, via the sensors 10, by the CCRAF system 40 (Step 120). The second set of measurement data can include, for example, immunocytochemistry data, including detection data and image data of proteins or other antigens. The second set of measurement can be collected for a multitude of samples, varying the process covariates, which must match the first set of measurements on a subset of the covariates (such as, for example, at some but not all time points).

Having received both the first set of measurement data (Step 310) and the second set of measurement data (Step 320), the CCRAF system 40 can train the machine learning (ML) platform (for example, an artificial neural network or ANN) comprising an encoder-decoder pairing (and encoder-decoder model) per set of measurement data, as well as joint embedding space (JES) model (Step 330). The ML platform can be trained by alternatively: (a) updating the encoder-decoder weights for each encoder-decoder model using an autoencoder objective by reconstructing its own set of measurements of all measurements (for example, as seen in FIG. 2 or 3, update the Encoder 1 and Decoder 1 as an autoencoder of the first set of measurement data); or (b) updating the encoder-decoder weights for each JES model using a predictive cross-encoder objective using samples where both the first set of measurement data and second set of measurement data are obtained (for example, as seen in FIG. 3, encoding the first set of measurement data using Encoder 1, decoding the embedding using Decoder 2, and updating the weights using a reconstruction loss on the second set of measurement data).

With the ML platform trained, the CCRAF system 40 can use the trained encoder-decoder models and JES model(s) for prediction tasks via the joint embedding (Step 340). In various embodiments, the CCRAF system 40 can perform prediction tasks such as, for example, but not limited to: predicting a detailed but difficult to acquirement measurement from a less detailed but easier to obtain measurement (for example, predict the immunocytochemistry output based on phase contrast images); predicting the measurements of a lab-scale vessel based on measurements of a bioprocess vessel, allowing reconstruction of richer measurements based on bioprocess measurements (such as, for example, time-series of temperature/pH/glucose/lactate/dissolved oxygen (DO)/etc); and forecasting future measurements based on early timepoints by training a forecasting model that predicts the future embedding based on the current one. Since the embedding is a highly compact abstract representation, it is a simpler machine learning task than learning to forecast based on the measurements directly. For example, the joint embedding is trained to capture the underlying biology shared between different measurement modalities and ignore measurement noise (for example, pixel noise in images).

Unless terminated (YES at Step 350), the process 300 will repeat (NO at Step 350).

To build and train for more than two modalities, the process 300 can include further sets of measurements following Steps 310 and 320. There is no strict need for all sets of measurements to overlap simultaneously, such as, for instance, all measured at the same timepoint. It can be sufficient for them to overlap pairwise, such as, for example: the first and second sets of measurements overlap at some time points; the second set of measurements overlap on some other time points with a third set of measurements; and the first and third sets of measurements overlap on further time points.

Although there is no strict requirement for every possible pairwise combination of measurements to exist, it is favorable if they do. Also, it is favorable if there are certain points where all have been measured, for example, some timepoint with all measurements.

In various embodiments, the CCRAF system 40 can be configured to constrain the joint embedding space by incorporating additional training objectives. For example, constraining the joint embedding space (or JES) model to a multivariate normal distribution by applying reparameterization and minimizing the Kullback Leibler-divergence between model embedding and a unit multivariate normal distribution according to the popular Variational Autoencoder (VAE) Framework.

In various other embodiments, the CCRAF system 40 can be configured to use data-specific architectures for the encoder and/or decoder imposing implicit priors to make model training easier. For example, using convolutional neural networks (CNNs) on image data.

In certain embodiments, the CCRAF system 40 can be used in control of stem cell differentiation. For example, the CCRAF 40 can use the JES model to inform control by making multiple predictions while varying the configuration of control parameters and measuring, via the sensors 10 (shown in FIG. 1), the effect on each prediction. Using this approach, it is possible to study the process in “what-if” scenarios. In the case of stem cell differentiation, it may be desirable to determine the optimal time to add a concentration of a molecule treatment to increase the yield of a target cell type. For instance, based on a current composition of the cell culture the CCRAF system 40 can forecast how the culture will change under different configuration of time and concentration and choose the configuration that results in the highest predicted yield.

In an embodiment, endpoint prediction can provide a basic opportunity to inform decision making. Predicting the endpoint yield allows optimization to increase endpoint yield, no more no less. Since the CCRAF system 40 can forecast multiple timesteps, it can allow decision making based on temporal evolution of the predicted parameter. For instance, by forecasting how the proportion of target cells increases over time, configurations can be selected that not only result in high yield, but that can reach high yields fast. This temporal information brings clear economic advantages from a protocol optimization perspective. For example, being able to shorten an 8-day protocol to an average of 7 days with sustained yield reduces the time which resources are required by 12.5% with similar decrease in medium use due to fewer medium changes required. In certain embodiments, the period of time can be shortened more than 12.5%.

In an embodiment, the CCRAF system 40 can be configured to represent and forecast stem cell differentiation processes and associated measurement data at a point in time in the future. Of note, the CCRAF 40 can be configured to maximize yield, as well as to optimize multiple factors such as speed of differentiation, viability, purity while minimizing line-to-line variability. The CCRAF system 40 can model the underlying biology of the processes under study and provide a flexible framework to predict between different sets of measurements and process conditions. It also provides a compact representation useful for forecasting biology that can be used to simulate what the future state of the cell culture will look like based on what is observed and measured at the current time point, and under different potential future conditions. Based on this forecast, better decisions can be made on how to adopt the protocol “on the fly”. If the CCRAF 40 simulates what will happen to the cell culture state under different simulated time points and concentrations of added growth factors, the timing and concentration can be adjusted to maximize the target objective. The CCRAF system 40 can simulate different protocol conditions based on initial conditions alone, allowing full in-silico screening of protocol parameters without experiment.

Learning a joint embedding by the CCRAF system 40 can help to inform how to run one experimental vessel or platform from another. By learning the joint embedding between a lab-scale vessel and bioprocess vessel, the JES model can provide better tools to optimize how to run bioprocesses of the same underlying cell culture process.

Forecasting using the JES model by the CCRAF system 40 allows early termination of differentiation experiments to save time and resources. Being able to forecast the future state of the cell culture, a determination can be made quickly regarding which experiments are unlikely to yield satisfactory results and terminate them early. This saves both experimental time as well as consumables.

In a nonlimiting application, the CCRAF system 40 can simulate the effect of added growth factors or small compounds based on initial conditions and planned protocol. Due to being simulation-based, many candidate protocols can be screened quickly, and the most promising ones experimentally run. Additionally, the CCRAF system 40 can provide an early readout of the protocol effect. During protocol development, many potential conditions can be screened, as well as how they influence differentiation results. Due to limited lab equipment, such screening experiments entail many rounds of experiments where a set of culture conditions are evaluated at any given time. Using in-silico screening of a large number of candidate protocols, as well as simulation based on real-time monitoring to achieve an earlier readout of its impact, for example 9 instead of 6 days, considerable amounts of time can be saved (time saved multiplied by the number of rounds required to finish), as well as consumables.

FIG. 8 shows a nonlimiting embodiment of the CCRAF system 40, constructed according to the principles of the disclosure. The CCRAF system 40 includes a processor 110, a machine learning (ML) platform 120, a storage 130, an interface suite 140, a simulator unit 150, and a communications unit 160. The CCRAF system 40 can include a bus (not shown) that can be connected to any or all of the components 110 to 160 by one or more communication links.

Any one or more of the components 110 to 160 can include a computing resource or a computing device. One or more of the components 120 to 160 can include a computing resource or a computing device that is separate from the processor 110, as seen in FIG. 2, or integrated with the processor 110. In certain embodiments, one or more of the components 120 and 140 to 160 can include a computer resource that can be executed on the processor 110 as one or more processes. The computer resources can be contained in the storage 130.

The bus can include any of several types of bus structures that can further interconnect to a memory bus (with or without a memory controller), a peripheral bus, and a local bus using any of a variety of commercially available bus architectures.

The processor 110 can include one or more processors, such as, for example, ab application specific integrated circuit (ASIC) and any of various commercially available processors, including for example, a central processing unit (CPU), a graphic processing unit (GPU), a general-purpose GPU (GPGPU), a dedicated neural processing unit (NPU), a tensor processing unit (TPU), a field programmable gate array (FGPA), an application-specific integrated circuit (ASIC), a system-on-a-chip (SOC), a single-board computer (SBC), a manycore processor, multiple microprocessors, or any other computing device architecture. The processor 110 can be arranged to interact with any of the components 120 to 160 to carry out or facilitate the processes included, described or contemplated by this disclosure. The processor 110 can be arranged to run one or more machine or deep learning systems.

The processor 110 can be arranged to run an operating system (OS), which can include an operating system (OS) kernel that can control all operations on the CCRAF system 40. The OS kernel can include, for example, a monolithic kernel or a microkernel. The OS kernel can be arranged to execute on the processor 110 and have control over operations in the processor 110.

The OS or OS kernel can be contained in the storage 130 and executed by the processor 110. The OS or OS kernel can be cached in the storage 130, such as, for example, in a random-access memory (RAM) 130B. The OS kernel can represent the highest level of privilege on the OS or the processor 110. The OS can include a driver for each hardware device with which the processor 110 might interact, including, for example, one or more receivers, transmitters, or transceivers in the communications unit 160. The OS kernel can be arranged to allocate resources or services to, and enable computing resources or processes to share or exchange information, protect the resources or services of each computing resource or process from other computing resources or processes, or enable synchronization amongst the computing resources or processes.

The OS kernel can, when a process is triggered, initiate and carry out the process for that computer resource, including allocating resources for the process, such as, for example, hard disk space, memory space, processing time or space, or other services on one or more hardware devices in the CCRAF system 40. The OS kernel can carry out the process by allocating memory space and processing resources to the process, loading the corresponding computing resource (or portion of a computing resource) into the allocated memory space, executing instructions of the computing resource on the OS kernel, or interfacing the process to one or more computer resources or processes.

The OS kernel can be arranged to facilitate interactions between the computing resources or processes. The processor 110, which runs the OS, can be arranged to arbitrate access to services and resources by the processes, including, for example, running time on the processor 110. The OS kernel can be arranged to take responsibility for deciding at any time which of one or more processes should be allocated to any of the resources.

The ML platform 120 can include a plurality of computer resources and/or computing devices. The ML platform 120 includes an encoder suite 120A having two or more encoders, a decoder suite 120B having two or more decoders, and a joint embedding space 120C, any one or more of which can include a computer resource and/or a computing device. The ML platform 120 can build and train a plurality of ML models, including a plurality of encoder-decoder models and at least one joint embedding space model. For example, each encoder (for example, Encoder 1, Encoder 2, etc.) in the encoder suite 120A can be paired with a respective decoder (for example, Decoder 1, Decoder 2, etc.) in the decoder suite 120B to build and train a respective encoder-decoder model. Further, the JES model can be built and trained by pairing an encoder (for example, Encoder 1) in the encoder suite 120A with a decoder (for example, Decoder 2) in the decoder suite 120B (as seen in FIG. 3), and joint embedding in the joint embedding space 120C to build and train the JES model. The joint embedding space 120C can facilitate comparisons between the plurality of sets of measurements input to the ML platform 120 for cross-modality retrieval.

The encoder suite 120A, the decoder suite 120B, and the joint embedding space 120C each include a plurality of computing resources, each arranged to run on the processor 110 or on one or more computing devices and interact the computer resources of the other components in the ML platform 120, as well as one or more of the components 130-160.

In various embodiments, the ML platform 120 includes a supervised ML platform, an unsupervised ML platform, or supervised and self-supervised ML platforms. The ML platform 120 includes a plurality of ML models built and trained to predict any combination of measurements for given cell culture based on a set of received measurement data. The ML models can be built and trained to forecast and simulate the future state of a cell culture, including future conditions and measurements of the cell culture.

The CCRAF system 40 can include a non-transitory computer-readable storage medium that can hold executable or interpretable computer resources, including computer program code or instructions that, when executed by the processor 110, cause the steps, processes or methods in this disclosure to be carried out, including building, training, testing, tuning and operation of the ML platform 120 and simulator unit 150. The computer-readable storage medium can be contained in the storage 130 or an external storage device (not shown).

The storage 130 can include a read-only memory (ROM) 130A, a random-access memory (RAM) 130B, a hard disk drive (HDD) 130C, and a database (DB) 130D. The storage 130 can provide nonvolatile storage of data, data structures, and computer-executable instructions, and can accommodate the storage of any data in a suitable digital format.

The storage 130 can include the non-transitory computer-readable medium that can hold the computer resources (including code or instructions) that can be executed (run) or interpreted by the operating system on the processor 110. The computer-readable medium can be contained in the HDD 130C.

A basic input-output system (BIOS) can be stored in the non-volatile memory in the ROM 130A, which can include, for example, an erasable programmable read-only memory (EPROM), or an electrically erasable programmable read-only memory (EEPROM). The BIOS can contain the basic routines that help to transfer information between any one or more of the components 110 to 160 in the CCRAF system 40, such as during start-up.

The RAM 130B can include a dynamic random-access memory (DRAM), a synchronous dynamic random-access memory (SDRAM), a static random-access memory (SRAM), a non-volatile random-access memory (NVRAM), or another high-speed RAM for caching data.

The HDD 130C can include, for example, an enhanced integrated drive electronics (EIDE) drive, a serial advanced technology attachments (SATA) drive, or any suitable hard disk drive for use with big data. The HDD can be configured for external use in a suitable chassis (not shown). The HDD can be arranged to connect to the bus B via a hard disk drive interface (not shown). In various nonlimiting embodiments, the HDD 130C can include the machine learning (ML) platform and/or the mechanistic platform.

The DB 130D can be arranged to be accessed by any one or more of the components in the system 300. The DB 130D can be arranged to receive a request and, in response, retrieve specific data, data records or portions of data records based on the request. A data record can include, for example, a file or a log. The DB 130D can include a database management system (DBMS) that can interact with the components 110 to 160. The DBMS can include, for example, SQL, NoSQL, MySQL, Oracle, Postgress, Access, or Unix. The DB 130D can include a relational database.

The DB 130D can be arranged to contain machine learning training datasets, testing datasets, and historical data. The DB 130D can contain information related to each cell culture, cell type, subtype, and fate.

Any number of computer resources can be stored in the storage 130, including, for example, a program module, an operating system (not shown), one or more application programs (not shown), or program data (not shown). Any (or all) of the operating system, application programs, program modules, and program data can be cached in the RAM as executable sections of computer code.

The network suite 140 includes an input/output (IO) interface 140A and a network interface 140B. The IO interface 140A can receive instructions or data from an operator via a human interface device (not shown), such as, for example, a keyboard (not shown), a mouse (not shown), a pointer (not shown), a stylus (not shown), a microphone (not shown), an interactive voice response (IVR) unit (not shown), a speaker (not shown), or a display device (not shown). The received instructions and data can be forwarded from the IO interface 140A as signals via one or more communication links to any component in the CCRAF system 40.

The network interface 140B can connect to the network 20 (shown in FIG. 1). The network interface 140B can be arranged to communicate with any number of devices (such as, for example, the sensors 10 or the computing device 30, shown in FIG. 1), either directly or via the network 20 over one or more communication links. The network interface 140B can include a wired or wireless communication network interface (not shown) or a wired or wireless modem (not shown). When used in a local area network (LAN), the network interface 140B can connect to the LAN network through the communication network interface; and, when used in a wide area network (WAN), it can connect to the WAN network through the modem. The modem (not shown) can be connected to the system bus via, for example, a serial port interface (not shown). The network interface 140B can be arranged to interact with the communications unit 160, or it can include a receiver (not shown), transmitter (not shown) or transceiver (not shown).

The simulator unit 150 can include one or more computer resources and/or computing devices, each configured to generate one or more multimedia signals that can be rendered by a human interface device, such as, for example, the display device of the computing device 30 (shown in FIG. 1). The multimedia signals can include audio and video signals that can be, for example, rendered by the human interface device, for example, as a displayed image or reproduced sound. The multimedia signals include measurements and time information. The measurements can include for example, an ML generated image of the cell culture, a forecasted yield for a target cell type, subtype, or fate, and the time point of the forecasted yield. The multimedia signals can include current cell state information, measurement data, and actions applied to, or performed on the cell culture.

The communications unit 160 can includes one or more transmitters, receivers, or transceivers. The communications 160 can be configured to communicate with sensor devices and computing devices, including the sensors 10 and computing device 30 (shown in FIG. 1).

The terms “a,” “an,” and “the,” as used in this disclosure, means “one or more,” unless expressly specified otherwise.

The term “backbone,” as used in this disclosure, means a transmission medium that interconnects one or more computing devices or communicating devices to provide a path that conveys data signals and instruction signals between the one or more computing devices or communicating devices. The backbone can include a bus or a network. The backbone can include an ethernet TCP/IP. The backbone can include a distributed backbone, a collapsed backbone, a parallel backbone or a serial backbone.

The term “bus,” as used in this disclosure, means any of several types of bus structures that can further interconnect to a memory bus (with or without a memory controller), a peripheral bus, or a local bus using any of a variety of commercially available bus architectures. The term “bus” can include a backbone.

The term “communication link,” as used in this disclosure, means a wired or wireless medium that conveys data or information between at least two points. The wired or wireless medium can include, for example, a metallic conductor link, a radio frequency (RF) communication link, an Infrared (IR) communication link, or an optical communication link. The RF communication link can include, for example, WiFi, WiMAX, IEEE 802.11, DECT, 0G, 1G, 2G, 3G, 4G, 5G, or 6G cellular standards, or Bluetooth. A communication link can include, for example, an RS-232, RS-422, RS-485, or any other suitable serial interface.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The terms “computer,” “computing device,” or “processor,” as used in this disclosure, means any machine, device, circuit, component, or module, or any system of machines, devices, circuits, components, or modules that are capable of manipulating data according to one or more instructions. The terms “computer,” “computing device” or “processor” can include, for example, without limitation, a communicating device, a computer resource, a processor, a microprocessor (μC), a central processing unit (CPU), a graphic processing unit (GPU), an application specific integrated circuit (ASIC), a general purpose computer, a super computer, a personal computer, a laptop computer, a palmtop computer, a notebook computer, a desktop computer, a workstation computer, a server, a server farm, a computer cloud, or an array or system of processors, μCs, CPUs, GPUs, ASICs, general purpose computers, super computers, personal computers, laptop computers, palmtop computers, notebook computers, desktop computers, workstation computers, or servers.

The terms “computing resource” or “computer resource,” as used in this disclosure, means software, a software application, a web application, a web page, a computer application, a computer program, computer code, machine executable instructions, firmware, or a process that can be arranged to execute on a computing device as one or more processes.

The term “computer-readable medium,” as used in this disclosure, means any non-transitory storage medium that participates in providing data (for example, instructions) that can be read by a computer. Such a medium can take many forms, including non-volatile media and volatile media. Non-volatile media can include, for example, optical or magnetic disks and other persistent memory. Volatile media can include dynamic random-access memory (DRAM). Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read. The computer-readable medium can include a “cloud,” which can include a distribution of files across multiple (e.g., thousands of) memory caches on multiple (e.g., thousands of) computers.

Various forms of computer readable media can be involved in carrying sequences of instructions to a computer. For example, sequences of instruction (i) can be delivered from a RAM to a processor, (ii) can be carried over a wireless transmission medium, or (iii) can be formatted according to numerous formats, standards or protocols, including, for example, WiFi, WiMAX, IEEE 802.11, DECT, 0G, 1G, 2G, 3G, 4G, 5G, or 6G cellular standards, or Bluetooth.

The term “database,” as used in this disclosure, means any combination of software or hardware, including at least one computing resource or at least one computer. The database can include a structured collection of records or data organized according to a database model, such as, for example, but not limited to at least one of a relational model, a hierarchical model, or a network model. The database can include a database management system application (DBMS). The at least one application may include, but is not limited to, a computing resource such as, for example, an application program that can accept connections to service requests from communicating devices by sending back responses to the devices. The database can be configured to run the at least one computing resource, often under heavy workloads, unattended, for extended periods of time with minimal or no human direction.

The terms “including,” “comprising” and their variations, as used in this disclosure, mean “including, but not limited to,” unless expressly specified otherwise.

The term “network,” as used in this disclosure means, but is not limited to, for example, at least one of a personal area network (PAN), a local area network (LAN), a wireless local area network (WLAN), a campus area network (CAN), a metropolitan area network (MAN), a wide area network (WAN), a metropolitan area network (MAN), a wide area network (WAN), a global area network (GAN), a broadband area network (BAN), a cellular network, a storage-area network (SAN), a system-area network, a passive optical local area network (POLAN), an enterprise private network (EPN), a virtual private network (VPN), the Internet, or the like, or any combination of the foregoing, any of which can be configured to communicate data via a wireless and/or a wired communication medium. These networks can run a variety of protocols, including, but not limited to, for example, Ethernet, IP, IPX, TCP, UDP, SPX, IP, IRC, HTTP, FTP, Telnet, SMTP, DNS, ARP, ICMP.

The term “server,” as used in this disclosure, means any combination of software or hardware, including at least one computing resource or at least one computer to perform services for connected communicating devices as part of a client-server architecture. The at least one server application can include, but is not limited to, a computing resource such as, for example, an application program that can accept connections to service requests from communicating devices by sending back responses to the devices. The server can be configured to run the at least one computing resource, often under heavy workloads, unattended, for extended periods of time with minimal or no human direction. The server can include a plurality of computers configured, with the at least one computing resource being divided among the computers depending upon the workload. For example, under light loading, the at least one computing resource can run on a single computer. However, under heavy loading, multiple computers can be required to run the at least one computing resource. The server, or any if its computers, can also be used as a workstation.

The terms “send,” “sent,” “transmission,” or “transmit,” as used in this disclosure, means the conveyance of data, data packets, computer instructions, or any other digital or analog information via electricity, acoustic waves, light waves or other electromagnetic emissions, such as those generated with communications in the radio frequency (RF) or infrared (IR) spectra. Transmission media for such transmissions can include coaxial cables, copper wire and fiber optics, including the wires that comprise a system bus coupled to the processor.

Devices that are in communication with each other need not be in continuous communication with each other unless expressly specified otherwise. In addition, devices that are in communication with each other may communicate directly or indirectly through one or more intermediaries.

Although process steps, method steps, or algorithms may be described in a sequential or a parallel order, such processes, methods and algorithms may be configured to work in alternate orders. In other words, any sequence or order of steps that may be described in a sequential order does not necessarily indicate a requirement that the steps be performed in that order; some steps may be performed simultaneously. Similarly, if a sequence or order of steps is described in a parallel (or simultaneous) order, such steps can be performed in a sequential order. The steps of the processes, methods or algorithms described in this specification may be performed in any order practical.

When a single device or article is described, it will be readily apparent that more than one device or article may be used in place of a single device or article. Similarly, where more than one device or article is described, it will be readily apparent that a single device or article may be used in place of the more than one device or article. The functionality or the features of a device may be alternatively embodied by one or more other devices which are not explicitly described as having such functionality or features.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

### EMBODIMENTS

Embodiment 1 is a computer-implemented method for cell culture representation based on two or more modalities, the method comprising:
receiving, by a processor, a first set of measurements of a cell culture, the first set of measurements being a first type of measurements;
applying, by the processor, the first set of measurements to a machine learning model;
predicting, by the machine learning model, a second set of measurements based on the first set of measurements, the second set of measurements being either the first type of measurements or a second type of measurements; and sending, by the processor, the second set of measurements to a computing device,
wherein the machine learning model is trained by:
   receiving, by the processor, at least two sets of training measurements of a cell culture, including a first set of training measurements of the first type and a second set of training measurements of the second type, with the second set of training measurements complementing the first set of training measurements; and
   training the machine learning model by an artificial neural network having a joint embedding space and a plurality of encoder-decoder pairings, including a first encoder-decoder pairing for the first set of training measurements and a second encoder-decoder for the second set of training measurements, wherein the first encoder-decoder pairing comprises a first encoder and a first decoder, and the second encoder-decoder pairing comprises a second encoder and a second decoder.

Embodiment 2 is the computer-implemented method of embodiment 1, wherein the training the model by the artificial neural network comprises updating encoder-decoder weights of the first encoder-decoder pairing using an autoencoder objective by reconstructing the first set of training measurements by the first encoder-decoder pairing.

Embodiment 3 is the computer-implemented method of embodiment 1, wherein the training the model by the artificial neural network comprises updating encoder-decoder weights using a predictive cross-encoder objective to encode the first set of training measurements by the first encoder, and decoding a joint embedding by the second decoder, wherein the updating the encoder-decoder weights is based at least in part on a reconstruction loss on the second set of training measurements.

Embodiment 4 is the computer-implemented method of embodiment 1, wherein the training the model by the artificial neural network comprises updating encoder-decoder weights using a predictive cross-encoder objective to encode the second set of training measurements by the second encoder, and decoding a joint embedding by the first decoder, wherein the updating the encoder-decoder weights is based at least in part on a reconstruction loss on the first set of training measurements.

Embodiment 5 is the computer-implemented method of embodiment 1, wherein the training the model by the artificial neural network comprises updating encoder-decoder weights of the second encoder-decoder pairing using an autoencoder objective by reconstructing the second set of training measurements by the second encoder-decoder pairing.

Embodiment 6 is the computer-implemented method of embodiment 1, wherein at least one of the first set of training measurements and/or the second set of training measurements comprises a measurement of the cell culture selected from the group consisting of a transmitted light microscopy image, a fluorescence microscopy image, a phase contrast microscopy image, differential interference contrast microscopy image, polarized light microscopy image, electron microscopy image, structured illumination microscopy image, a pH measurement, a glucose measurement, a lactate measurement, a temperature measurement, a pressure measurement, a dissolved oxygen measurement, a spectroscopy measurement, a conductivity measurement, an optical density measurement, a capacitance measurement, a viscosity measurement, a redox potential measurement, a mass spectroscopy measurement, an ultrasound-based measurement of fluid density, a nutrient measurement, an -omics measurement (such as, e.g., metabolomics, genomics, epigenomics, lipidomics, glycomics, transcriptomics, and proteomics), and a combination thereof.

Embodiment 7 is the computer-implemented method of embodiment 1, wherein the cell culture is a stem cell culture undergoing differentiation.

Embodiment 8 is the computer-implemented method of embodiment 7, wherein the stem cell culture is selected from an embryonic stem cell culture, an adult stem cell culture, an induced pluripotent stem cell culture, or a trophoblast stem cell culture.

Embodiment 9 is the computer-implemented method of embodiment 7, wherein the stem cell culture is a mesenchymal stem cell culture, a hematopoietic stem cell culture, a neural stem cell culture, an epithelial stem cell culture, or a cord blood stem cell culture.

Embodiment 10 is the computer-implemented method of embodiment 7, wherein the stem cell culture comprises progenitor cells.

Embodiment 11 is the computer-implemented method of embodiment 10, wherein the progenitor cells are selected from the group consisting of mesodermal progenitor cells, endodermal progenitor cells, ectodermal progenitor cells, neural progenitor cells, cardiac progenitor cells, hematopoietic progenitor cells, mesenchymal stem cells, pancreatic progenitor cells, and a combination thereof.

Embodiment 12 is the computer-implemented method of embodiment 7, wherein the stem cell culture undergoing differentiation results in the stem cell culture differentiating into a mesoderm, endoderm, and/or ectoderm.

Embodiment 13 is the computer-implemented method of embodiment 12, wherein the mesoderm comprises a skeletal muscle cell, a kidney cell, a red blood cell, or a smooth muscle cell.

Embodiment 14 is the computer-implemented method of embodiment 12, wherein the endoderm comprises a lung cell, a thyroid cell, or a pancreatic cell.

Embodiment 15 is the computer-implemented method of embodiment 12, wherein the ectoderm comprises a skin cell, a neuron cell, or a pigment cell.

Embodiment 16 is the computer-implemented method of embodiment 1, wherein the machine learning model comprises a joint embedded space model.

Embodiment 17 is the computer-implemented method of any one of embodiments 1 to 16, wherein training the machine learning model by the artificial neural network comprises applying a temporal smoothing loss to joint embedding to ensure there is a smooth transition between embeddings of measurements taken at nearby time points.

Embodiment 18 is the computer-implemented method of any one of embodiments 1 to 17, wherein training the machine learning model by the artificial neural network comprises applying a regularization to joint embedding to ensure that the embedding is constrained to a multivariate probability distinction.

Embodiment 19 is a non-transitory computer readable storage medium storing one or more programs, the one or more programs comprising instructions, which, when executed by a processor, perform:
receiving a first set of measurements of a cell culture, the first set of measurements being a first type of measurements;
applying the first set of measurements to a machine learning model;
predicting a second set of measurements based on the first set of measurements, the second set of measurements being either the first type of measurements or a second type of measurements; and
sending the second set of measurements to a computing device,
wherein the machine learning model is trained by:
   receiving, by the processor, at least two sets of training measurements of a cell culture, including a first set of training measurements of the first type and a second set of training measurements of the second type, with the second set of training measurements complementing the first set of training measurements; and
   training the machine learning model by an artificial neural network having a joint embedding space and a plurality of encoder-decoder pairings, including a first encoder-decoder pairing for the first set of training measurements and a second encoder-decoder for the second set of training measurements, wherein the first encoder-decoder pairing comprises a first encoder and a first decoder, and the second encoder-decoder pairing comprises a second encoder and a second decoder.

Embodiment 20 is the non-transitory computer readable storage medium of embodiment 19, wherein the training the model by the artificial neural network comprises updating encoder-decoder weights of the first encoder-decoder pairing using an autoencoder objective by reconstructing the first set of training measurements by the first encoder-decoder pairing.

Embodiment 21 is the non-transitory computer readable storage medium of embodiment 19, wherein the training the model by the artificial neural network comprises updating encoder-decoder weights using a predictive cross-encoder objective to encode the first set of training measurements by the first encoder, and decoding a joint embedding by the second decoder, wherein the updating the encoder-decoder weights is based at least in part on a reconstruction loss on the second set of training measurements.

Embodiment 22 is the non-transitory computer readable storage medium of embodiment 19, wherein the training the model by the artificial neural network comprises updating encoder-decoder weights using a predictive cross-encoder objective to encode the second set of training measurements by the second encoder, and decoding a joint embedding by the first decoder, wherein the updating the encoder-decoder weights is based at least in part on a reconstruction loss on the first set of training measurements.

Embodiment 23 is the non-transitory computer readable storage medium of embodiment 19, wherein the training the model by the artificial neural network comprises updating encoder-decoder weights of the second encoder-decoder pairing using an autoencoder objective by reconstructing the second set of training measurements by the second encoder-decoder pairing.

Embodiment 24 is the non-transitory computer readable storage medium of embodiment 19, wherein at least one of the first set of training measurements and/or the second set of training measurements comprises a measurement of the cell culture selected from the group consisting of a transmitted light microscopy image, a fluorescence microscopy image, a phase contrast microscopy image, differential interference contrast microscopy image, polarized light microscopy image, electron microscopy image, structured illumination microscopy image, a pH measurement, a glucose measurement, a lactate measurement, a temperature measurement, a pressure measurement, a dissolved oxygen measurement, a spectroscopy measurement, a conductivity measurement, an optical density measurement, a capacitance measurement, a viscosity measurement, a redox potential measurement, a mass spectroscopy measurement, an ultrasound-based measurement of fluid density, a nutrient measurement, an -omics measurement (such as, e.g., metabolomics, genomics, epigenomics, lipidomics, glycomics, transcriptomics, and proteomics), and a combination thereof.

Embodiment 25 is the non-transitory computer readable storage medium of embodiment 19, wherein the cell culture is a stem cell culture undergoing differentiation.

Embodiment 26 is the non-transitory computer readable storage medium of embodiment 19, wherein the stem cell culture is selected from an embryonic stem cell culture, an adult stem cell culture, an induced pluripotent stem cell culture, or a trophoblast stem cell culture.

Embodiment 27 is the non-transitory computer readable storage medium of embodiment 19, wherein the stem cell culture is a mesenchymal stem cell culture, a hematopoietic stem cell culture, a neural stem cell culture, an epithelial stem cell culture, or a cord blood stem cell culture.

Embodiment 28 is the non-transitory computer readable storage medium of embodiment 19, wherein the stem cell culture comprises progenitor cells.

Embodiment 29 is the non-transitory computer readable storage medium of embodiment 28, wherein the progenitor cells are selected from the group consisting of mesodermal progenitor cells, endodermal progenitor cells, ectodermal progenitor cells, neural progenitor cells, cardiac progenitor cells, hematopoietic progenitor cells, mesenchymal stem cells, pancreatic progenitor cells, and a combination thereof.

Embodiment 30 is the non-transitory computer readable storage medium of embodiment 19, wherein the stem cell culture undergoing differentiation results in the stem cell culture differentiating into a mesoderm, endoderm, and/or ectoderm.

Embodiment 31 is the non-transitory computer readable storage medium of embodiment 30, wherein the mesoderm comprises a skeletal muscle cell, a kidney cell, a red blood cell, or a smooth muscle cell.

Embodiment 32 is the non-transitory computer readable storage medium of embodiment 30, wherein the endoderm comprises a lung cell, a thyroid cell, or a pancreatic cell.

Embodiment 33 is the non-transitory computer readable storage medium of embodiment 30, wherein the ectoderm comprises a skin cell, a neuron cell, or a pigment cell.

Embodiment 34 is the non-transitory computer readable storage medium of embodiment 19, wherein the machine learning model comprises a joint embedded space model.

Embodiment 35 is the non-transitory computer readable storage medium of any one of embodiments 19 to 34, wherein training the machine learning model by the artificial neural network comprises applying a temporal smoothing loss to joint embedding to ensure there is a smooth transition between embeddings of measurements taken at nearby time points.

Embodiment 36 is the non-transitory computer readable storage medium of any one of embodiments 19 to 35, wherein training the machine learning model by the artificial neural network comprises applying a regularization to joint embedding to ensure that the embedding is constrained to a multivariate probability distinction.

Embodiment 37 is a system for cell culture representation based on two or more modalities, the system comprising:
a memory;
a communication unit configured to receive requests from, or send forecasting measurements to, a computing device; and
a processor, wherein the processor is configured to:
   receive at least two sets of training measurements of a cell culture, including a first set of training measurements of the first type and a second set of training measurements of the second type, with the second set of training measurements complementing the first set of training measurements; and
   train a machine learning model by an artificial neural network having a joint embedding space and a plurality of encoder-decoder pairings, including a first encoder-decoder pairing for the first set of training measurements and a second encoder-decoder for the second set of training measurements, wherein the first encoder-decoder pairing comprises a first encoder and a first decoder, and the second encoder-decoder pairing comprises a second encoder and a second decoder.

Embodiment 38 is the system of embodiment 37, wherein the training the model by the artificial neural network comprises updating encoder-decoder weights of the first encoder-decoder pairing using an autoencoder objective by reconstructing the first set of training measurements by the first encoder-decoder pairing.

Embodiment 39 is the system of embodiment 37, wherein the training the model by the artificial neural network comprises updating encoder-decoder weights using a predictive cross-encoder objective to encode the first set of training measurements by the first encoder, and decoding a joint embedding by the second decoder, wherein the updating the encoder-decoder weights is based at least in part on a reconstruction loss on the second set of training measurements.

Embodiment 40 is the system of embodiment 37, wherein the training the model by the artificial neural network comprises updating encoder-decoder weights using a predictive cross-encoder objective to encode the second set of training measurements by the second encoder, and decoding a joint embedding by the first decoder, wherein the updating the encoder-decoder weights is based at least in part on a reconstruction loss on the first set of training measurements.

Embodiment 41 is the system of embodiment 37, wherein the training the model by the artificial neural network comprises updating encoder-decoder weights of the second encoder-decoder pairing using an autoencoder objective by reconstructing the second set of training measurements by the second encoder-decoder pairing.

Embodiment 42 is the system of embodiment 37, wherein at least one of the first set of training measurements and/or the second set of training measurements comprises a measurement of the cell culture selected from the group consisting of a transmitted light microscopy image, a fluorescence microscopy image, a phase contrast microscopy image, differential interference contrast microscopy image, polarized light microscopy image, electron microscopy image, structured illumination microscopy image, a pH measurement, a glucose measurement, a lactate measurement, a temperature measurement, a pressure measurement, a dissolved oxygen measurement, a spectroscopy measurement, a conductivity measurement, an optical density measurement, a capacitance measurement, a viscosity measurement, a redox potential measurement, a mass spectroscopy measurement, an ultrasound-based measurement of fluid density, a nutrient measurement, an -omics measurement (such as, e.g., metabolomics, genomics, epigenomics, lipidomics, glycomics, transcriptomics, and proteomics), and a combination thereof.

Embodiment 43 is the system of embodiment 37, wherein the cell culture is a stem cell culture undergoing differentiation.

Embodiment 44 is the system of embodiment 37, wherein the stem cell culture is selected from an embryonic stem cell culture, an adult stem cell culture, an induced pluripotent stem cell culture, or a trophoblast stem cell culture.

Embodiment 45 is the system of embodiment 37, wherein the stem cell culture is a mesenchymal stem cell culture, a hematopoietic stem cell culture, a neural stem cell culture, an epithelial stem cell culture, or a cord blood stem cell culture.

Embodiment 46 is the system of embodiment 37, wherein the stem cell culture comprises progenitor cells.

Embodiment 47 is the system of embodiment 46, wherein the progenitor cells are selected from the group consisting of mesodermal progenitor cells, endodermal progenitor cells, ectodermal progenitor cells, neural progenitor cells, cardiac progenitor cells, hematopoietic progenitor cells, mesenchymal stem cells, pancreatic progenitor cells, and a combination thereof.

Embodiment 48 is the system of embodiment 37, wherein the stem cell culture undergoing differentiation results in the stem cell culture differentiating into a mesoderm, endoderm, and/or ectoderm.

Embodiment 49 is the system of embodiment 48, wherein the mesoderm comprises a skeletal muscle cell, a kidney cell, a red blood cell, or a smooth muscle cell.

Embodiment 50 is the system of embodiment 48, wherein the endoderm comprises a lung cell, a thyroid cell, or a pancreatic cell.

Embodiment 51 is the system of embodiment 48, wherein the ectoderm comprises a skin cell, a neuron cell, or a pigment cell.

Embodiment 52 is the system of embodiment 37, wherein the machine learning model comprises a joint embedded space model.

Embodiment 53 is the system of embodiment 37, wherein the artificial neural network comprises:
an encoder suite comprising a plurality of encoders, including the first encoder and the second encoder.

Embodiment 54 is the system of embodiment 37, wherein the artificial neural network comprises:
a decoder suite comprising a plurality of decoders, including the first decoder and the second decoder.

Embodiment 55 is the system of embodiments 53 and 54, wherein the number encoders in the encoder suite equals the number decoders in the decoder suite.

Embodiment 56 is the system of embodiments 53 and 54, wherein:
the joint embedding space is configured to receive encoded measurement data from each of the plurality of encoders; and
each of the plurality of decoders is configured to receive joint embedded data from the joint embedding space to reconstruct at least one of the first set of training data and the second set of training data.

Embodiment 57 is the system of any of embodiments 37 to 56, wherein training the machine learning model by the artificial neural network comprises applying a temporal smoothing loss to joint embedding to ensure there is a smooth transition between embeddings of measurements taken at nearby time points.

Embodiment 58 is the system of any one of embodiments 37 to 57, wherein training the machine learning model by the artificial neural network comprises applying a regularization to joint embedding to ensure that the embedding is constrained to a multivariate probability distinction.

Embodiment 59 is a computer-implemented method for predicting downstream measurements in a cell culture, the method comprising:
receiving, by a processor, cell culture measurement data;
applying, by the processor, the cell culture measurement data to a head machine learning model;
predicting, by the head machine learning model, measurements in the cell culture at a future time based on the applied cell culture measurement data; and
sending, by the processor, the predicted measurements to a computing device.

Embodiment 60 is the computer-implemented method of embodiment 59, wherein the head machine learning model is trained by previously:
receiving prior cell culture measurement data for said cell culture or a different cell culture;
applying the prior cell culture measurement data to an encoder trained to learn numerical representations of cell culture process data;
mapping by the trained encoder the prior cell culture measurement data into an embedding space, including a tensor representation providing a representation of cell culture biology, to provide embedded cell culture measurement data; and
applying the embedded cell culture measurement data as an input to, and downstream measurement data as an output from, the head machine learning model.

Embodiment 61 is the computer-implemented method of embodiment 60, wherein the encoder operates according to a trained joint embedding space (JES) model.

Embodiment 62 is the computer-implemented method of embodiment 60 or 61, wherein the prior culture measurement data is matched 1:1 with measurements of a process stage downstream of the cell culture process.

Embodiment 63 is the computer-implemented method of any of embodiments 60 to 62, wherein parametric values of the encoder are frozen during training of the head machine learning model.

### EXAMPLES

### Example 1: Case Studies for Prediction and Forecasting

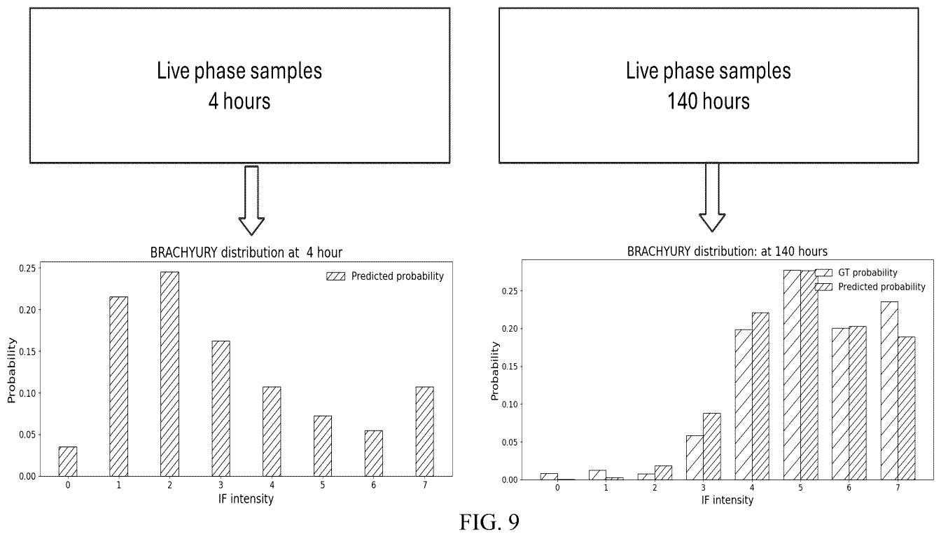

Here, an application of the approach described herein to a simple baseline approach was compared. Two modalities of data were used that describe stem cell populations during differentiation through time: live phase contrast (PC) images (modality 1) and histograms of pixel intensities of fixed immunofluorescence (IF) images (modality 2). In IF images, relatively high pixel intensities typically corresponded to cells considered positive, while relatively low pixel intensities corresponded to cells considered negative for the corresponding marker that was imaged. Thus, if the distributions of pixel intensities of certain markers of interest can be predicted, the distribution of the corresponding cell population states can be understood. This was achieved by converting the IF images into normalized histograms of pixel intensities (probability mass functions) with 10 bins, each corresponding to a 10th quantile of all observed pixel intensities. The goal of the approaches presented in this case study was to predict and forecast modality 2 using modality 1 on a holdout test set (FIG. 9). The Jensen-Shannon (JS) divergence (JSD—a normalized value between 0 and 1) was used on the predicted and ground truth probability mass functions to evaluate the approaches.

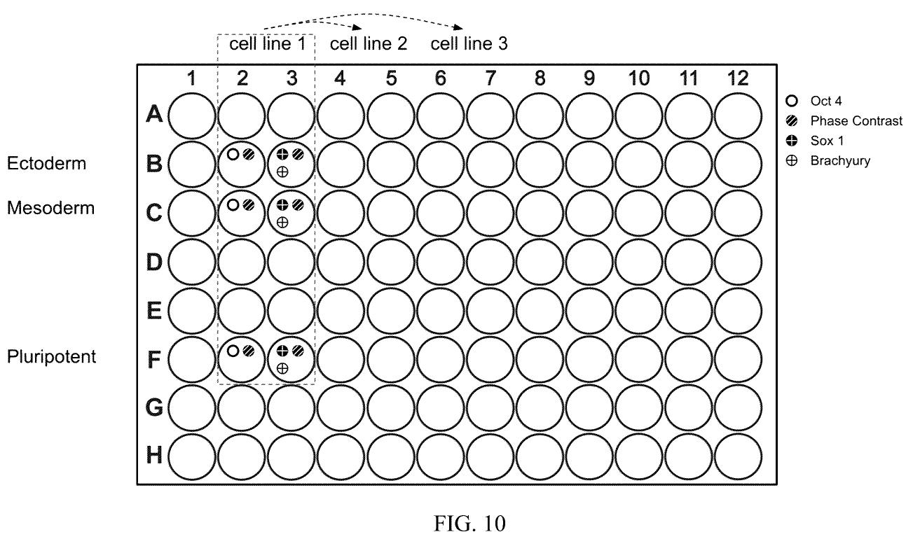

This approach was sought to be validated in two ways: (1) first, to predict modality 2 measured at time point to using modality 1 also measured at time point to (Case 1); and (2) second, to predict modality 2 at any future time point tₙ₊ₖ using modality 1 measured at time point to (Case 2). These tasks required a dataset with PC and IF measurements at multiple time points, where the covariates are aligned at some of the time points, but not all. Accordingly, an experiment was designed with three different stem cell lines that were triggered to differentiate towards either ectoderm, mesoderm, or to stay in pluripotent conditions over six days. A replicate plate (FIG. 10) was created using these experimental conditions for each day (six plates in total) so that the cells were fixed at the end of each 24 hours and stained using IF markers. PC images of the cell populations were captured every 4 hours, and IF images were used to measure the expression levels of markers SOX1 (positive for ectoderm), BRACHYURY (positive for mesoderm), and OCT4 (positive for pluripotency) at the end of each day. Thus, an IF measurement of the (fixed) cell state every 24 hours in combination with phase contrast images every four hours of live cells was provided. For testing the models, cross validation across three different cell lines was used, using two of the cell lines for training and the remaining one for validation.

### Case 1: Prediction and Forecasting

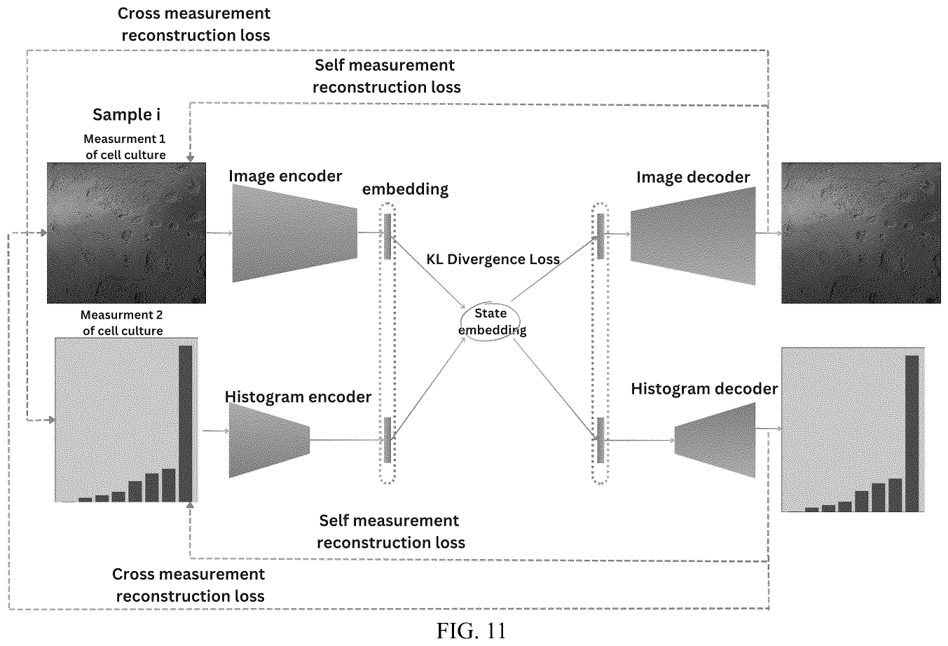

To model the two modalities, two paired encoder-decoder architectures represented by convolutional neural networks were deployed. The embedding space was constrained by a variational loss, specifically using a multi-modal Variational Autoencoder (VAE) framework. The KL divergence was computed between the prior and the multi-modal posterior over the embedding space, incorporating the entropy of the embedding posterior. For each modality, a reconstruction loss was applied, ensuring accurate reconstruction from modality 1 to modality 1 and from modality 2 to modality 2. Additionally, when the covariates of modality 1 and modality 2 were aligned, a cross-reconstruction loss was introduced, enabling reconstruction of modality 2 from modality 1 and vice versa (FIG. 11).

The training task for prediction involves predicting the target (modality 2) at time point (tₙ) given the input (modality 1) at the same time point (tₙ).

The training task for forecasting involves predicting the target (modality 2) at a future time point (tₙ₊₄ₕ) given the input (modality 1) at time point (tₙ). The forecasting time resolution was discretized by the modality with the highest time resolution, which was the four-hour time resolution of modality 1 in this case. Temporal encoder was included to account for time covariate.

To benchmark the approach for forecasting, it was evaluated against a typical baseline approach that consisted of an ImageNet pretrained ResNet18 (convolutional neural network). A weighted cross entropy loss in combination with an Adam optimizer was used to train the network.

The baseline approach was limited to using the aligned modality samples only during training. This means that models could be trained to predict 1 up to 6 days into the future at the same time, but now the forecasting time resolution was discretized by the modality with the lowest time resolution, which was the 24-hour time resolution of modality 2 in this case. Here, the baseline can only predict modality 2, 24 hours into the future given an input image from modality 1.

### Results:

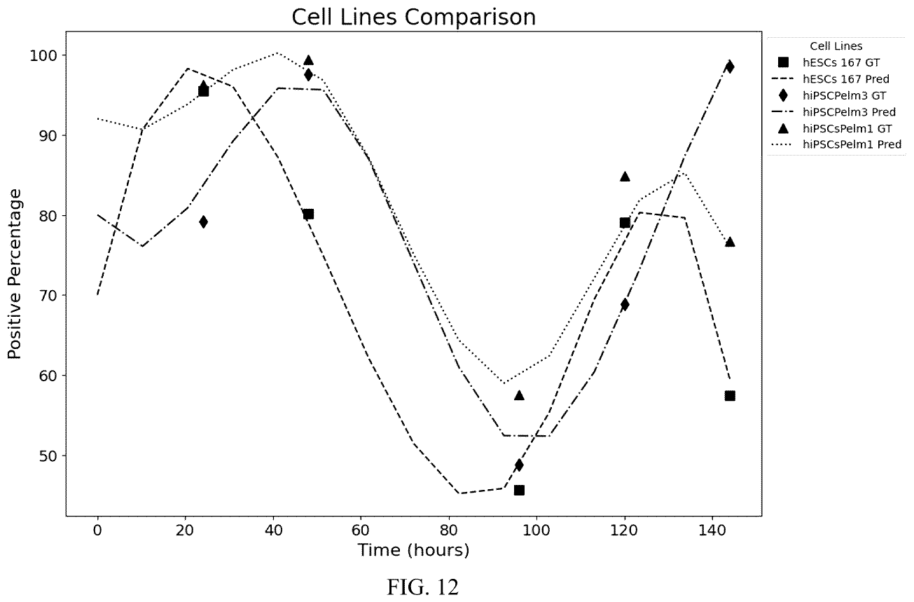

In Table 1, the prediction error of modality 2 from modality 1 was presented using JS divergence. In addition to comparing the predicted and target histograms directly, thresholds of the histograms were also used to get the percentage of cells that were positive for a given marker. Thresholds were defined for each of the markers separately by imaging experts. The predicted percentage of positive cells was then computed for a given marker using modality 1, allowing to model the dynamic of these percentages at a much higher time resolution at a fraction of the cost and effort than would be required if this were done using IF imaging (modality 2). Cross validation on all cell lines was performed, and it was shown that the approach closely follows the ground truth measurements for all cell lines over time (FIG. 12).

TABLE 1
Overview of JS divergence scores on the holdout cell line between
the predicted and ground truth normalized histograms (modality
2) on average and stratified by target marker. Lower is better.

| JS average | JS Brachyury | JS Sox1 | JS Oct4 |
|---|---|---|---|
| 0.003 | 0.004 | 0.001 | 0.003 |

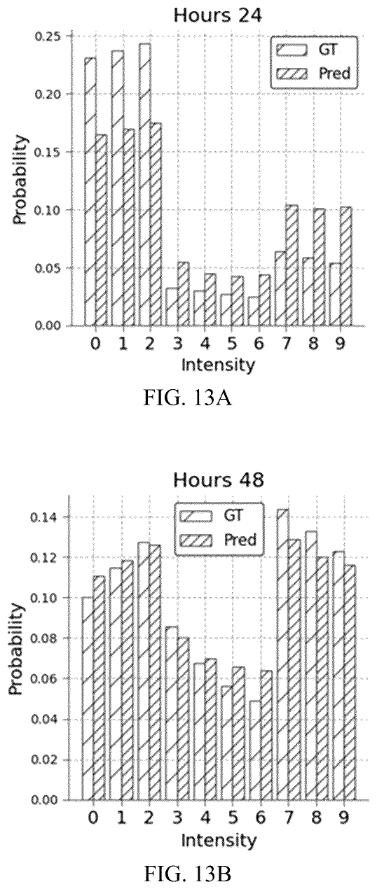

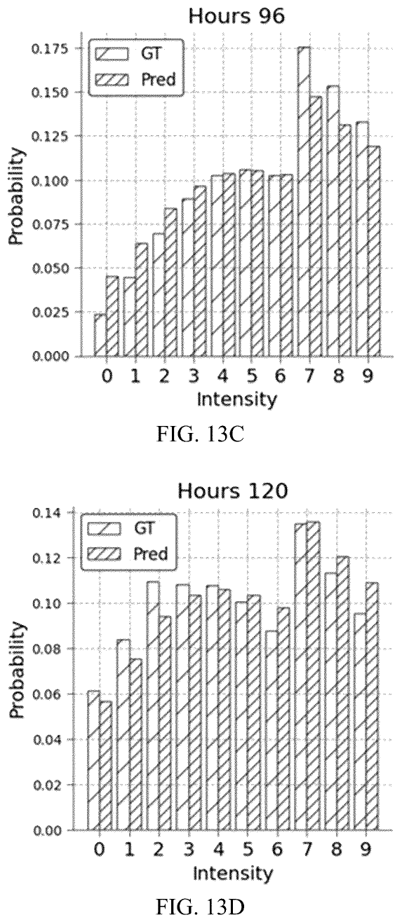

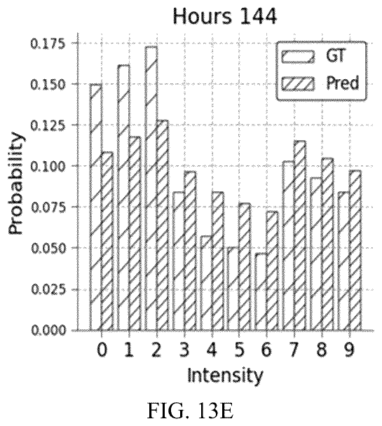

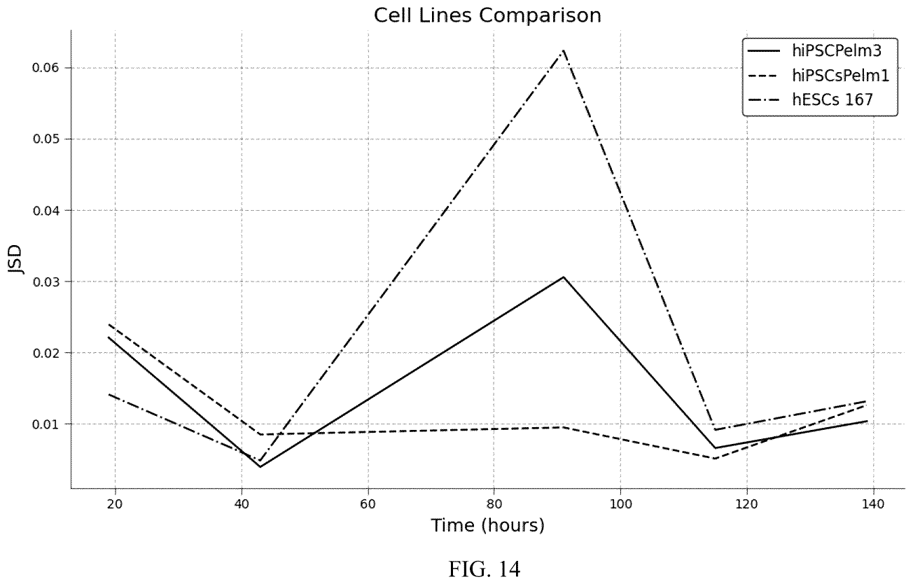

The experimental approach demonstrates better performance (Table 2) and more flexibility in terms of time resolution than the baseline approach, allowing to forecast modality 2 (and modality 1) indefinitely in four-hour time steps compared to the fixed number of forecasting steps at a lower time resolution from the baseline approach (FIGS. 13A-13E and 14).

TABLE 2
Overview of JS divergence scores on the holdout cell line between
the forecasted and ground truth normalized histograms (modality
2) on average by Brachyury target marker. Lower is better.

| Method | JS Brachyury |
|---|---|
| Our approach | 0.009 |
| Baseline | 0.184 |

### Example 2: Case Study for Benchmarking Method on Forecasting Future Biomarker Expression

The purpose of this case study is to benchmark the invention method on forecasting future biomarker expression. The case study describes a specific embodiment of the claim DMVAE methodology, where the specific below is referred to as dynamic full experts multimodal mixtures of experts (DFE-MMoE). It is described in a scientific paper submitted to the AAAI conference 2025 (attached).

TABLE 3
Data and Experimental Setup

| Item | Details |
|---|---|
| Stem-cell lines | Commercially available human stem cell lines: hESC 167, hiPSC |
| | Pelm3, hiPSC Pelm1 |
| Cell culture | 6 days of directed differentiation towards either ectoderm or |
| conditions | mesoderm lineage, or maintenance of pluripotency (6 days) |
| Modality 1 (non- | Phase-contrast microscopy, 20x, every 4 hours |
| destructive) | |
| Modality 2 | Immunofluorescence (IF) staining of OCT4, SOX1, BRACHYURY |
| (destructive) | every 24 hours |
| Representation of IF | 10-bin pixel-intensity histograms (probability mass functions) |
| Forecasting task | From the latest 3 PC frames predict the IF histogram 12 hours ahead |
| Score | Symmetric Kullback-Leibler (KL) divergence (distance between two |
| | distributions; 0 = identical) |
| Validation | Leave-one-cell-line-out, 3 folds |

TABLE 4
Models Compared

| | | Missing-data | |
|---|---|---|---|
| Model | What it is | policy | Temporal logic |
| DFE-MMOE | Separate encoders per | Handles any | Forward + backward |
| (method of | modality | subset (e.g. IF | dynamics during training; |
| instant invention) | Mixture-of-Experts¹ | missing 23 hours/ | forward recursion during |
| | fusion into a latent space | day) | inference |
| | Bidirectional LSTM | | |
| | models past + future | | |
| | embeddings during | | |
| | training | | |
| CNN-LSTM | ResNet-18 encoder + | Needs aligned PC + | Forward recursion only² |
| baseline | LSTM decoder per | IF | |
| | marker | | |

Footnotes:
¹each modality produces its own distribution over latent space; these are combined by a learnable weighted average, giving graceful degradation if a modality is absent;
²at inference time the model predicts step t + 1 from all information up to step t, then re-uses that prediction to get step t + 2, etc. (No look-ahead once the run begins).

TABLE 5
Implementation Details

| Component | DMVAE settings | CNN-LSTM settings |
|---|---|---|
| PC encoder | ResNet-18 trunk (ImageNet weights) | same |
| IF encoder | Three 2-layer MLPs (one per marker) | N/A |
| Dynamic | 1-layer LSTM over 3-step sequences | 1-layer LSTM |
| encoder/decoder | | |
| Losses | Reconstruction: IF × 1.0, PC × 0.01 | Cross-entropy on |
| | KL term (β = 1.0) | histograms |
| Optimiser | Adam, LR = 1 × 10⁻⁴ | same |
| Hardware | Single NVIDIA T4; total training time ≈ 7 | same |
| | hours/fold | |

Training schedule: 100 epochs multimodal pre-training (PC↔IF), then 4×50 epochs joint dynamic training (see Algorithm 1 in paper for additional details).

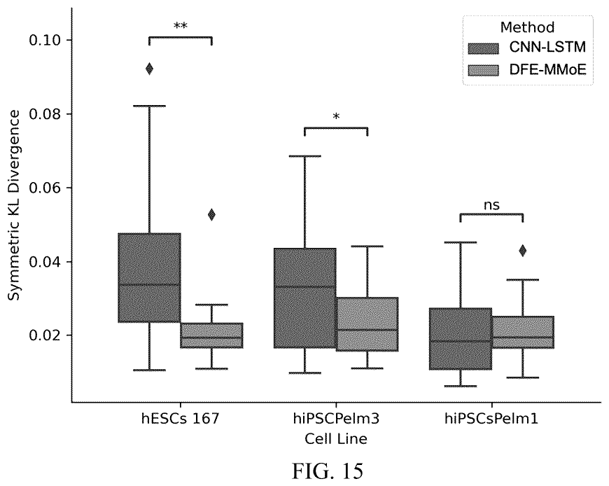

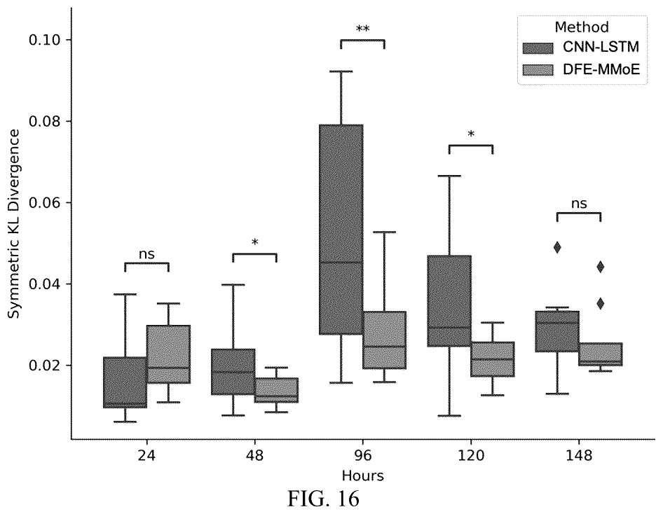

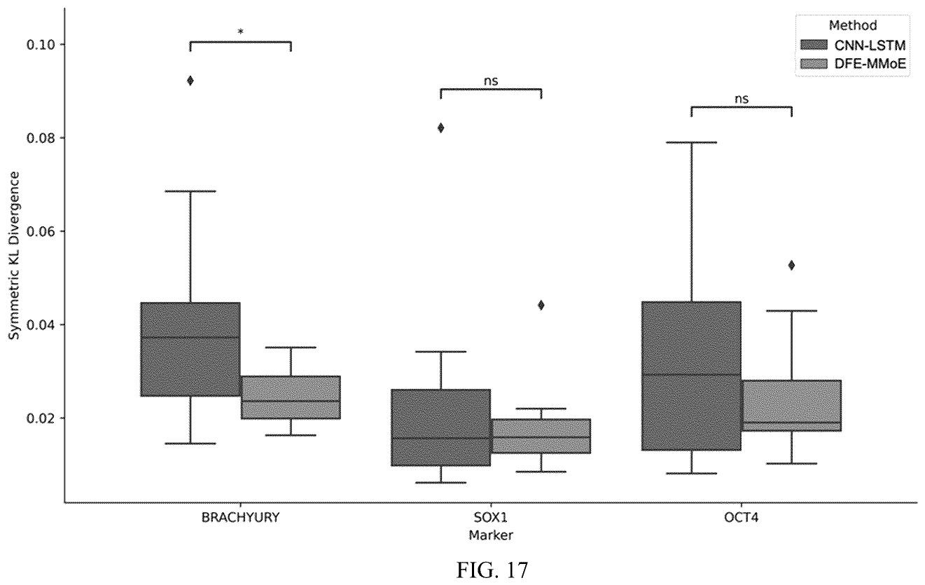

Results: DFE-MMoE demonstrates superior and more consistent performance compared to the CNN-LSTM model across various covariates as demonstrated by cell line (FIG. 15), by culture age (FIG. 16), and by marker (FIG. 17).

Benefits: The proposed approach achieves lower forecasting error across cell lines, over culture time, as well as per marker, signifying a clear benefit of the DMVAE methodology compared to the supervised CNN-LSTM baseline. The model learns from every 4-hour phase-contrast frame, even when 24-hour IF stains are missing, thanks to its mixture-of-experts fusion and variational training, so it gives manufacturers earlier, more reliable go/no-go signals while reducing batch loss. Running inference in under one second per well on a single commodity GPU/CPU, it slots easily into inline quality-control or closed-loop media-optimization pipelines, and its modality-agnostic design means new sensors (e.g., transcriptomics) can be added without architectural re-work.

1. A computer-implemented method for cell culture representation based on two or more modalities, the method comprising:
receiving, by a processor, a first set of measurements of a cell culture, the first set of measurements being a first type of measurements;
applying, by the processor, the first set of measurements to a machine learning model;
predicting, by the machine learning model, a second set of measurements based on the first set of measurements, the second set of measurements being either the first type of measurements or a second type of measurements; and
sending, by the processor, the second set of measurements to a computing device,
wherein the machine learning model is trained by:
receiving, by the processor, at least two sets of training measurements of a cell culture, including a first set of training measurements of the first type and a second set of training measurements of the second type, with the second set of training measurements complementing the first set of training measurements; and
training the machine learning model by an artificial neural network having a joint embedding space and a plurality of encoder-decoder pairings, including a first encoder-decoder pairing for the first set of training measurements and a second encoder-decoder for the second set of training measurements, wherein the first encoder-decoder pairing comprises a first encoder and a first decoder, and the second encoder-decoder pairing comprises a second encoder and a second decoder.

2. The computer-implemented method of claim 1, wherein the training the model by the artificial neural network comprises:
(1) updating encoder-decoder weights of the first encoder-decoder pairing using an autoencoder objective by reconstructing the first set of training measurements by the first encoder-decoder pairing; and/or
(2) updating encoder-decoder weights of the second encoder-decoder pairing using an autoencoder objective by reconstructing the second set of training measurements by the second encoder-decoder pairing.

3. The computer-implemented method of claim 1, wherein the training the model by the artificial neural network comprises updating encoder-decoder weights using a predictive cross-encoder objective to encode the first set of training measurements by the first encoder, and decoding a joint embedding by the second decoder, wherein the updating the encoder-decoder weights is based at least in part on a reconstruction loss on the second set of training measurements.

4. The computer-implemented method of claim 1, wherein the training the model by the artificial neural network comprises updating encoder-decoder weights using a predictive cross-encoder objective to encode the second set of training measurements by the second encoder, and decoding a joint embedding by the first decoder, wherein the updating the encoder-decoder weights is based at least in part on a reconstruction loss on the first set of training measurements.

5. (canceled)

6. The computer-implemented method of claim 1, wherein at least one of the first set of training measurements and/or the second set of training measurements comprises a measurement of the cell culture selected from the group consisting of a transmitted light microscopy image, a fluorescence microscopy image, a phase contrast microscopy image, differential interference contrast microscopy image, polarized light microscopy image, electron microscopy image, structured illumination microscopy image, a pH measurement, a glucose measurement, a lactate measurement, a temperature measurement, a pressure measurement, a dissolved oxygen measurement, a spectroscopy measurement, a conductivity measurement, an optical density measurement, a capacitance measurement, a viscosity measurement, a redox potential measurement, a mass spectroscopy measurement, an ultrasound-based measurement of fluid density, a nutrient measurement, an -omics measurement (such as, e.g., metabolomics, genomics, epigenomics, lipidomics, glycomics, transcriptomics, and proteomics), and a combination thereof.

7-15. (canceled)

16. The computer-implemented method of claim 1, wherein the machine learning model comprises a joint embedded space model.

17. The computer-implemented method of claim 1, wherein training the machine learning model by the artificial neural network comprises applying a temporal smoothing loss to joint embedding to ensure there is a smooth transition between embeddings of measurements taken at nearby time points.

18. The computer-implemented method of claim 1, wherein training the machine learning model by the artificial neural network comprises applying a regularization to joint embedding to ensure that the embedding is constrained to a multivariate probability distinction.

19. A non-transitory computer readable storage medium storing one or more programs, the one or more programs comprising instructions, which, when executed by a processor, perform:
receiving a first set of measurements of a cell culture, the first set of measurements being a first type of measurements;
applying the first set of measurements to a machine learning model;
predicting a second set of measurements based on the first set of measurements, the second set of measurements being either the first type of measurements or a second type of measurements; and
sending the second set of measurements to a computing device,
wherein the machine learning model is trained by:
receiving, by the processor, at least two sets of training measurements of a cell culture, including a first set of training measurements of the first type and a second set of training measurements of the second type, with the second set of training measurements complementing the first set of training measurements; and
training the machine learning model by an artificial neural network having a joint embedding space and a plurality of encoder-decoder pairings, including a first encoder-decoder pairing for the first set of training measurements and a second encoder-decoder for the second set of training measurements, wherein the first encoder-decoder pairing comprises a first encoder and a first decoder, and the second encoder-decoder pairing comprises a second encoder and a second decoder.

20-36. (canceled)

37. A system for cell culture representation based on two or more modalities, the system comprising:
a memory;
a communication unit configured to receive requests from, or send forecasting measurements to, a computing device; and
a processor, wherein the processor is configured to:
receive at least two sets of training measurements of a cell culture, including a first set of training measurements of the first type and a second set of training measurements of the second type, with the second set of training measurements complementing the first set of training measurements; and
train a machine learning model by an artificial neural network having a joint embedding space and a plurality of encoder-decoder pairings, including a first encoder-decoder pairing for the first set of training measurements and a second encoder-decoder for the second set of training measurements, wherein the first encoder-decoder pairing comprises a first encoder and a first decoder, and the second encoder-decoder pairing comprises a second encoder and a second decoder.

38. The system of claim 37, wherein the training the model by the artificial neural network comprises;
(1) updating encoder-decoder weights of the first encoder-decoder pairing using an autoencoder objective by reconstructing the first set of training measurements by the first encoder-decoder pairing; and/or
(2) updating encoder-decoder weights of the second encoder-decoder pairing using an autoencoder objective by reconstructing the second set of training measurements by the second encoder-decoder pairing.

39. The system of claim 37, wherein the training the model by the artificial neural network comprises updating encoder-decoder weights using a predictive cross-encoder objective to encode the first set of training measurements by the first encoder, and decoding a joint embedding by the second decoder, wherein the updating the encoder-decoder weights is based at least in part on a reconstruction loss on the second set of training measurements.

40. The system of claim 37, wherein the training the model by the artificial neural network comprises updating encoder-decoder weights using a predictive cross-encoder objective to encode the second set of training measurements by the second encoder, and decoding a joint embedding by the first decoder, wherein the updating the encoder-decoder weights is based at least in part on a reconstruction loss on the first set of training measurements.

41. (canceled)

42. The system of claim 37, wherein at least one of the first set of training measurements and/or the second set of training measurements comprises a measurement of the cell culture selected from the group consisting of a transmitted light microscopy image, a fluorescence microscopy image, a phase contrast microscopy image, differential interference contrast microscopy image, polarized light microscopy image, electron microscopy image, structured illumination microscopy image, a pH measurement, a glucose measurement, a lactate measurement, a temperature measurement, a pressure measurement, a dissolved oxygen measurement, a spectroscopy measurement, a conductivity measurement, an optical density measurement, a capacitance measurement, a viscosity measurement, a redox potential measurement, a mass spectroscopy measurement, an ultrasound-based measurement of fluid density, a nutrient measurement, an -omics measurement (such as, e.g., metabolomics, genomics, epigenomics, lipidomics, glycomics, transcriptomics, and proteomics), and a combination thereof.

43-51. (canceled)

52. The system of claim 37, wherein the machine learning model comprises a joint embedded space model.

53-56. (canceled)

57. The system of claim 37, wherein training the machine learning model by the artificial neural network comprises;
(1) applying a temporal smoothing loss to joint embedding to ensure there is a smooth transition between embeddings of measurements taken at nearby time points; and/or
(2) applying a regularization to joint embedding to ensure that the embedding is constrained to a multivariate probability distinction.

58. (canceled)

59. A computer-implemented method for predicting downstream measurements in a cell culture, the method comprising:
receiving, by a processor, cell culture measurement data;
applying, by the processor, the cell culture measurement data to a head machine learning model;
predicting, by the head machine learning model, measurements in the cell culture at a future time based on the applied cell culture measurement data; and
sending, by the processor, the predicted measurements to a computing device.

60. The computer-implemented method of claim 59, wherein the head machine learning model is trained by previously:
receiving prior cell culture measurement data for said cell culture or a different cell culture;
applying the prior cell culture measurement data to an encoder trained to learn numerical representations of cell culture process data;
mapping by the trained encoder the prior cell culture measurement data into an embedding space, including a tensor representation providing a representation of cell culture biology, to provide embedded cell culture measurement data; and
applying the embedded cell culture measurement data as an input to, and downstream measurement data as an output from, the head machine learning model.

61. The computer-implemented method of claim 60, wherein the encoder operates according to a trained joint embedding space (JES) model.

62. (canceled)

63. The computer-implemented method of claim 60, wherein parametric values of the encoder are frozen during training of the head machine learning model.
